(19) United States
(12) Patent Application Publication
(10) Pub. No.: US 2023/0263587 A1
(43) Pub. Date: Aug. 24, 2023

(54) SYSTEMS AND METHODS FOR PREDICTING AND PREVENTING BLEEDING AND OTHER ADVERSE EVENTS

(71) Applicant: Wayne State University, Detroit, MI (US)

(72) Inventors: Abhilash K. Pandya, Grosse Ile, MI (US); Mostafa Daneshgar Rahbar, Dearborn Heights, MI (US); Luke A. Reisner, Madison Heights, MI (US); Hao Ying, Novi, MI (US)

(73) Assignee: Wayne State University, Detroit, MI (US)

(21) Appl. No.: 18/028,150

(22) PCT Filed: Sep. 22, 2021
(86) PCT No.: PCT/US2021/051607

§ 371 Date: Mar. 23, 2023

Related U.S. Application Data
(60) Provisional application No. 63/082,464, filed on Sep. 23, 2020.

(51) Int. Cl. A61B 90/00; A61B 34/20; A61B 34/37

(52) U.S. Cl. CPC A61B 90/03; A61B 34/20; A61B 34/37; A61B 2034/2065; A61B 2090/032

(57) ABSTRACT

Various systems, methods, and devices for identifying instrument movements likely to cause intraoperative bleeding are described. An example method includes identifying a movement of an instrument; determining that the movement of the instrument exceeds a threshold; and dampening the movement of the instrument based on determining that the movement exceeds the threshold.

### CROSS-REFERENCE TO RELATED APPLICATION

This application claims the priority of U.S. Provisional App. No. 63/082,464, which was filed on Sep. 23, 2020 and is incorporated by reference herein in its entirety.

### BACKGROUND

Intraoperative bleeding is a major complication of minimally invasive surgeries that negatively impacts surgical outcomes. Bleeding can be caused by accidental damage to the arteries or veins of the patient and may be related to surgical skills. Penza, V., et al, FRONTIERS IN ROBOTICS AND AI, 2017, 4: p. 15. Other causes of bleeding include anatomical anomalies or disorders, recent intake of drugs, or hemostasis disorders (which may be either congenital or acquired). Curnow, J., et al, THE SURGERY J., 2016, 2(01): p. e29-e43.

If a surgeon does not detect and address bleeding complications quickly, these complications may result in the death of the patient. Intraoperative bleeding is a major cause of death during the surgical process. Philips, P.A, et al., J. OF THE AM. COL. OF SURGEONS, 2001, 192(4): p. 525-536. According to the 2004 Nationwide Inpatient Sample database, 2.23 million (or 5.8%) patients in the United States required transfusions to address complications related to bleeding. Morton, J., et al., AM. J. OF MED. QUALITY, 2010, 25(4): p. 289-296. On average, patients receiving transfusions due to bleeding complications were 1.7 times more likely to die, 1.9 times more likely to develop an infection, stayed in the hospital 2.5 times longer, and had treatment costs that were $17,194 higher than their counterparts with no bleeding complications.

Intraoperative bleeding is a critical yet difficult problem to manage during various types of surgical procedures. Controlling patient bleeding during procedures that are already complex can be challenging for surgeons. Bleeding is of particular significance in robotic-assisted surgery. The overall complication rate of robotic-assisted surgery ranges from 4.3% to 12.0%. Patel, V.R., et al., JOURNAL OF ENDOUROLOGY, 2008, 22(10): p. 2299-2306; Jeong, J., et al., J. OF ENDOUROLOGY, 2010, 24(9): p. 1457-1461; Lebeau, T., et al., SURGICAL ENDOSCOPY, 2011, 25(2): p. 536-542. Bleeding is difficult to manage in minimally invasive (either robotic or traditional laparoscopic) surgery, where the surgeon completes the procedure using a remote camera view. In these cases, a small bleed can quickly lead to visual occlusion of part or the entire camera view. To effectively address bleeding, the surgeon continually monitors the camera view for bleeding to rapidly estimate the source. This estimation is particularly difficult because the source of bleeding is often submerged in, or otherwise occluded in, a pool of blood (or can quickly become submerged). Traditionally, the choices for the surgeon are limited. In cases wherein the surgeon proceeds blindly, the surgeon can potentially cause more damage. Strategies to clear blood from the camera view, such as using suction to clear blood, may cause more bleeding from torn vessels and other damage. If the camera must be removed from the patient cavity for cleaning, this results in a loss of position and orientation relative to the bleed and may cause additional delays.

In addition to the risks of bleeding to patients, bleeding complications cause other problems. Hospitals and insurance providers must bear the costs associated with the numerous problems that arise from surgical bleeding. For example, it is necessary to purchase tools used for the management of bleeding, pay the staff required to treat the affected patients, and manage the recovery rooms that these patients must occupy for prolonged periods following surgery due to complications from intraoperative bleeding. Hospitals have a tremendous need to minimize the resources spent on the management of intraoperative bleeding. The detection and localization of bleeding during surgery, particularly in the case of arterial bleeding, have the potential to reduce intraoperative complexity and patient blood loss.

Schafer et al. conducted a research study on intraoperative bleeding complications during robotic surgery. Schäfer, M., et al., THE AM. J. OF SURGERY, 2000, 180(1): p. 73-77. The authors concluded that, in all, 331 (2.3%) of 14,391 patients had intraoperative bleeding complications. Moreover, 44 patients (13.3%) suffered from external bleeding of the abdominal wall, whereas the remaining 287 patients (86.7%) suffered from internal bleeding. It was noted that 33 patients (10.0%) with internal bleeding received blood transfusions, and the patients had a mean blood loss of 1,630 milliliters. Surgical hemostasis was performed in 68.0% of external bleeds and 91.0% of internal bleeds. There were 250 patients (1.8%) with postoperative bleeding complications. External bleeding occurred in 143 patients, and 107 patients developed internal bleeding. Special treatment was used in 92.0% of the cases of external bleeding. Further surgical intervention was required in half of the cases of internal bleeding. Major vascular injuries occurred in 12 patients (0.1%), with open treatment being necessary in all cases reviewed. Bleeding complications are common during laparoscopic surgery. In order to effectively manage bleeding complications, meticulous dissection techniques, immediate recognition of bleeding region, and adequate surgical treatment can help manage bleeding complications,

Tensions exerted on the tissues of the patient, the unprepared cutting of arterial vessels, and accidental movements made by the surgeon are three sources of sudden bleeding during robotic and laparoscopic surgeries, which are related to the lack of experience of the surgeon. Shafaat et al. considered arterial bleeding to be one of the most significant complications that can occur during robotic surgery, requiring immediate compression or clamping to remedy the blood flow. Talab, S.S., et al., J. OF UROLOGY, 2019, 201(Supplement 4): p. e851. The fear of bleeding is one of the factors that discourages surgeons from undertaking a minimally invasive approach. Novellis, P., et al., ANNALS OF CARDIOTHORACIC SURGERY, 2019, 8(2): p. 292. Hemorrhaging is the second most common complication in laparoscopic surgery, with incidents occurring in 0.2% to 1.1% of laparoscopic surgeries. Castillo, O.A., et al., SURGICAL LAPAROSCOPY ENDOSCOPY & PERCUTANEOUS TECHNIQUES, 2008, 18(3): p. 315-18. This represents a challenging and/or intimidating situation for any laparoscopic surgeon. Barros, M.B., et al., SAO PAULO MED. J., 2005, 123(1): pp. 38-41. Garisto et al. argued that strategies for managing intraoperative bleeding complications during robotic surgery could allow the safe utilization of robotic techniques in renal tumor surgery. Garisto, J., et al., J. OF UROLOGY, 2019, 201(Supplement 4): p. e848. However, an important limitation of minimally invasive surgical procedures is the loss of real-time endoscopic visualization when hemorrhaging is inadvertently caused (also known as the “red-out” situation), such as in cases where bleeding occurs following obtaining a tumor biopsy sample. Ishihara, R., et al., GASTROINTESTINAL ENDOSCOPY, 2008, 68(5): pp. 975-81.

It has also been shown that arterial bleeding can lead to intraoperative catastrophes. Intraoperative catastrophes can be considered as events that require unplanned surgical procedures, such as an emergency thoracotomy. Cao, C., et al., ANNALS OF THORACIC SURGERY, 2019. For example, in a study of 1,810 patients that underwent robotic anatomical pulmonary resections, the most common catastrophic event was intraoperative hemorrhaging from the pulmonary artery. Other common catastrophic events included injury to the airway, the pulmonary vein, and the liver. Cao, et al. Management of sudden bleeding situations can save time and resources both for patients and the healthcare system. However, such management depends on early detection of bleeding, especially during robotic and laparoscopic surgery, mainly before blood obscures the surgeon’s vision. This early detection can help the operational team to prevent the situation from turning into a red-out and to localize and visualize the source of bleeding in the event that a red-out occurs.

Preventable medical errors in the operating room cost many human lives every year in the United States and around the world. These preventable medical errors are classified as adverse events in the medical field. An adverse event can be defined as “an unintended injury or complication resulting in prolonged length of hospital stay disability at the time of discharge or death caused by healthcare management and not by the patients’ underlying disease” (Brennan, T.A., et al., NEJM, 1991. 324(6): p. 370-376). Adverse events cause potentially preventable patient harm, lengthen hospital stays, and increase health care costs. See Andrews, L.B., et al., LANCET, 1997. 349(9048): p. 309-313; Thomas, E.J., et al., COSTS OF MED. INJURIES IN UTAH AND COLORADO. INQUIRY, 1999: p. 255-264. Many in-hospital adverse events are associated with surgical care. de Vries, E.N., et al., BMJ QUALITY & SAFETY, 2008. 17(3): pp. 216-23.

Efforts to improve patient safety can target leading causes of potentially preventable patient harm, which can be identified from the frequency, severity, and preventability of adverse events. Aranaz-Andrés, J.M., et al., INT’L J. FOR QUALITY IN HEALTH CARE, 2009. 21(6): pp. 408-14. Anderson et al. included fourteen record review studies incorporating 16,424 surgical patients in their systematic review. They concluded that adverse events occurred in 14.4% of patients (interquartile range [IQR], 12.5% to 20.1%), and potentially preventable adverse events occurred in 5.2% (IQR, 4.2% to 7.0%). The consequences of 3.6% of adverse events (IQR, 3.1% to 4.4%) were fatal, those of 10.4% (IQR, 8.5% to 12.3%) were severe, those of 34.2% (IQR, 29.2% to 39.2%) were moderate, and those of 52.5% (IQR, 49.8% to 55.3%) were minor. Anderson, O., et al., AM. J. OF SURGERY, 2013. 206(2): pp. 253-62. Preventable and potentially preventable errors still occur in the treatment of severely injured patients. Errors in hemorrhage control and airway management are common human treatment errors. Id.

Many factors can lead to safety issues during robotic surgeries. Human error is one aspect, such as wrong commands or manipulation. Although these errors are inversely proportional to the experience of a surgeon, there is still room for human error. One major source of those human errors in the field of robotic surgery is related to the use of the manipulator arms. In minimally invasive surgery, the robot is operated in teleoperation mode, where the surgeon controls the robot manipulator arms with hand controllers. Melinek, J., et al., J. OF FORENSIC SCIENCES, 2004. 49(5): p. JFS2003218-5. Risky movement of surgical tools near critical tissue can potentially lead to unexpected bleeding, tissue damage, or other adverse events during the surgery. In contrast to open surgery, surgeons do not have a full field of view in robotic surgery. Sometimes this can be a source of a lack of situational awareness and can lead to the sharpness of movements of robotic tools due to a lock of complete visualization. Moreover, sometimes they cannot precisely adjust the level of force that is exerted by the manipulation arm as there is no force feedback of the tools. This can also produce abrupt movements of surgical tools. A related source of abrupt movement is that due to a lack of force feedback, when a tool gets tangled or stuck in tissue and is manipulated, it can cause a rubber banding or springing of the tool due to lack of appropriate force feedback to the user.

Robotic and other minimally invasive surgical techniques are performed with the endoscopic camera and instruments passed percutaneously placed through small ports. Unlike open surgeries, the prevention of unexpected bleeding and tissue damage is more crucial in minimally invasive surgery.

### SUMMARY

An example method includes identifying a movement of an instrument; determining that the movement of the instrument exceeds a threshold; and dampening the movement of the instrument based on determining that the movement exceeds the threshold.

In some examples, the method is performed by a system including at least one processor. For example, the processor may be executing instructions stored in memory. In some cases, the system is included in a robotic surgical system.

### BRIEF DESCRIPTION OF THE DRAWINGS

FIG. 1 illustrates an example environment for predicting intraoperative bleeding.

FIG. 2 illustrates example techniques for generating entropy pixels representing the entropy of pixels in frames depicting a scene of interest.

FIG. 3 illustrates an example of a technique for predicting bleeding based on entropy maps.

FIG. 4 illustrates an example of a modified frame indicating a dangerous movement by highlighting.

FIG. 5 illustrates an example of a modified frame indicating a dangerous movement by pop-up notifications.

FIG. 6 illustrates a process for preventing intraoperative bleeding in a robotic surgical environment.

FIG. 7 illustrates a process for preventing intraoperative bleeding in a surgical environment.

FIG. 8 illustrates an example of a system configured to perform various functions described herein.

FIG. 9 illustrates a flow chart of a sample process that can be used to identify and locate bleeding, such as arterial bleeding.

FIG. 10 depicts a change in a surgery scene at a location of arterial bleeding.

FIG. 11 shows a result of Example 2 following import into Adobe Premier software.

FIG. 12 depicts the temporal entropy within a prerecorded video with arterial bleeding. It can be seen that the first abrupt change in the number of red pixels in the entropy map occurred in frame 95. The right graph is the zoomed version of the left one in the neighborhood of frame number 95. It shows that identifying abrupt changes in the number of red pixels within the local entropy map can be used to detect arterial bleeding within the surgery scene.

FIG. 13 depicts an entropy map of a surgery scene before arterial bleeding, as well as an entropy map including two types of pixels at the moment of arterial bleeding.

FIG. 14 includes two images demonstrate the effect of arterial bleeding at two time points within a surgery scene. Gray shapes in FIG. 14 represent pixels corresponding to bleeding in the surgical scene. Black shapes in FIG. 14 represent pixels corresponding to tool movement in the surgical scene.

FIG. 15 includes three images comparing the change in the Fourier Transform of the surgery scene.

FIG. 16 illustrates an example of a technique for importing the recorded video into video editing software.

FIG. 17 illustrates a graph showing a change of entropy due to surgical tool movement during a surgery.

### DETAILED DESCRIPTION

This disclosure describes approaches for predicting bleeding, prior to occurrence of the bleeding. Bleeding can be predicted, for instance, based on spatio-temporal entropy and/or patterns in kinematic data associated with a surgical robot. For example, bleeding may be predicted when a movement (or an input directing movement) of a surgical tool is at a particular velocity, acceleration, jerk, or a combination thereof that is consistent with causing bleeding. In some cases, the predicted bleeding is prevented by outputting an indication based on the predicted bleeding and/or dampening the movement of surgical tools based on the predicted bleeding.

A warning system to inform the surgeon about such movements is a key component for the development of any intelligent assistant for robotic surgery. Unexpected bleeding and tissue damage can be prevented by relating risky movement of surgical tools within the normal procedure (excluding suturing, which typically requires fast tool movement) to the likelihood of occurrence of unexpected bleeding (or other adverse events) and tissue damage. Furthermore, this information can be used to mitigate the risky movements of manipulator arms. Various implementations described herein provide real-time feedback to the surgeon about the manner in which the surgeon uses the surgical tools.

A computer-based intelligent system that passively observes and proactively warns a surgeon during surgery in real time can help prevent issues such as unexpected damage to tissue and vessels during surgery. This system is preventative, leveraging a link between unexpected bleeding/tissue damage and the usage of surgical tools. This system can be especially useful for a less experienced surgeon, such as a resident, a fellow, or a surgeon inexperienced with robotic surgical tools. When combined with a surgical robot, the system can be able to proactively prevent or mitigate unsafe tool movements. If the system predicts or detects unsafe tool movement, the system may alter the movement of the corresponding robotic arm to smooth the motion, reduce the force or velocity, or even halt a dangerous motion.

There is a need for information that will enable surgeons to more effectively and safely move surgical tools during minimally invasive surgery. Example systems described herein provide information about the movements that need to be corrected by localizing the risky movement. Many existing systems rely completely on the level of experience of the surgeon. There is no tool to monitor, inform, and assist the surgeon during the surgery. In contrast, the examples described herein provide real-time tools for quantifying and assessing the surgeon’s performance during surgery based on his/her usage of surgical instruments.

Current surgical practice and the commercial market are focused on managing complications caused by human error and misuse of tools (such as tissue damage/bleeding) after they occur. This means that dangerous situations that potentially put a patient’s life at risk are not prevented when they could be. Focusing on managing complications, rather than preventing complications, can add significant costs to the healthcare system. In contrast, various systems described herein use imaging and algorithms to help prevent unsafe movements before damage is done. Thus, example systems can assist the surgeon by warning them about and mitigating risky movements of surgical tools. Various examples can also be used to quantify the performance of surgeons based on their usage of surgical tools in real time during robotic surgery.

During robotic and laparoscopic surgeries, vascular injuries may occur as a result of accidental instrument movements or cutting of the vessels, which may lead to arterial bleeding. The detection and localization of arterial bleeding during robotic or laparoscopic surgeries is critical to overcoming complications associated with intraoperative hemorrhaging. If this sudden bleeding is not detected and controlled in a timely manner, it can lead to a “red-out” situation, where blood spreads throughout the surgical scene, leading to the occlusion of the surgeon’s field of view. This disclosure describes vision-based techniques for monitoring abrupt changes within the surgical scene to predict bleeding (e.g., arterial bleeding).

There is a demand for tracking systems of surgical tool movements for open surgery as well as minimally invasive surgery to warn the surgeon about their risky movements during the surgery. An example tracking system can be utilized to assess surgical movements during open surgery and minimally invasive surgery. Open surgery is still the standard of care for many areas of interventional medicine. Unfortunately, it also is the most difficult paradigm to measure because the surgeon freely manipulates several different tools. One approach has been to track the surgeon’s hands. Commercial gloves with embedded sensors can be worn by the surgeon to track each joint position and velocity of the 23-DOF of each surgical hand. Reiley, C.E., et al., SURGICAL ENDOSCOPY, 2011, 25(2): pp. 356-366. There are a variety of commercially available wireless data gloves, such as Cyberglove II from CYBERGLOVE SYSTEMS LLC of San Jose, CA; ShapeWrap II from MEASURAND INC.; and products from FIFTH DIMENSION TECHNOLOGIES, INC. Reiley, C.E., et al., SURGICAL ENDOSCOPY, 2011, 25(2): pp. 356-66. The Advanced Dundee Endoscopic Psychomotor Tester (ADEPT) uses an optical motion tracking system to compare the performance of expert and novice surgeons. Francis, N.K., et al., ARCHIVES OF SURGERY, 2002, 137(7): pp. 841-44. Infrared light is reflected off sensors placed on a surgeon’s arm. The positional data of the markers are extrapolated via trajectory analysis. However, as an optical tracking system, ADEPT suffers from line of sight issues and can result in omitted data. Also, limbs cannot be simultaneously tracked because of overlapping signals. These issues are an obstacle to the acceptance of ADEPT in the operating room.

The Imperial College Surgical Assessment Device (ICSAD) instead uses electromagnetic markers placed on the dorsal side of each hand and the Isotrak II system (Polhemus, USA) to track the surgeon’s hand movements in open surgical simulated tasks. The ICSAD records basic motion metrics at a rate of 20 Hz, such as the number and speed of hand movements, distance traveled, and total task time. This technology has been useful in analyzing several laparoscopic and open tasks. Datta, V., et al., J. OF THE AM. COL. OF SURGEONS, 2001, 193(5): pp. 479-485; Taffinder, N., et al., SURG. ENDOSC, 1999. 13(suppl 1), p. 81. However, the ICSAD is limited to ex vivo benchtop models because the extraneous wires or markers cannot be used on the surface of gloves in live surgery.

Robot-assisted minimally invasive surgery provides a rich source of motion information to be used for analysis. For example, the da Vinci surgical system from INTUITIVE SURGICAL, INC. of Sunnyvale, CA, can provide position and velocity information for the robotic joints, high-resolution stereoscopic video, and other system status variables with an activated Application Programming Interface (API). This data from the da Vinci system is sent over a communication line at 23 Hz and stored on a local computer. This method of data collection using synchronized video and kinematic data is promising, because there are no hardware modifications necessary to track movements and the operation of the robot is not affected. Force information is currently not available from the da Vinci system. Adding sensors to track position or record forces on instruments directly is not feasible for robotic and traditional minimally invasive instruments because of the high costs and time associated with embedding the sensors, the limited number of uses, and sterilization issues. Thompson, C.A., JCAHO GEARS UP TO SURVEY STERILE COMPOUNDING PRACTICES, 2004, Oxford University Press.

Overall, current methods employed for tracking the motion of surgical tools include instrumented tools, measurements of the surgeon’s arm kinematics and force (Lin, H.C., et al., COMPUTER AIDED SURGERY, 2006, 11(5): pp. 220-230; Rosen, J., et al., IEEE TRANSACTIONS ON BIOMEDICAL ENGINEERING, 2006. 53(3): pp. 399-413), and physical and virtual simulators (Pugh, C.M. and P. Youngblood, J. OF THE AM. MED. INFORMATICS ASS’N, 2002. 9(5): pp. 448-60). Also, there are a variety of promising new avenues of research, including vision-based techniques to track instruments with distinct colored markers (see, e.g., Ko, S.-Y. and D.-S. Kwon, A surgical knowledge based interaction method for a laparoscopic assistant robot in RO-MAN 2004. 13th IEEE INTERNATIONAL WORKSHOP ON ROBOT AND HUMAN INTERACTIVE COMMUNICATION (2004)) and eye-gaze patterns (see, e.g., JA, I., et al., Gaze patterns in laparoscopic surgery, MEDICINE MEETS VIRTUAL REALITY: THE CONVERGENCE OF PHYSICAL & INFORMATIONAL TECHNOLOGIES: OPTIONS FOR A NEW ERA IN HEALTHCARE (1999)).

Analyzing motion can be broken into two main categories: (1) dexterity analysis through generating descriptive statistics that provide intuition about the data; or (2) building more structured time series (language) models of the data to gain insight into understanding what the surgeon is doing assessment for the different surgical paradigms. See Reiley, C.E., et al., SURGICAL ENDOSCOPY, 2011, 25(2): pp. 356-66.

Dexterity analysis can create descriptive statistics using recorded motions of the system or forces exerted on the surgical environment. Common metrics include motion of the instrument, motion economy, peak forces, and torques (see, e.g., Rosen, J., et al., IEEE TRANSACTIONS ON BIOMEDICAL ENGINEERING, 2006, 53(3): pp. 399-413, Yamauchi, Y., et al. Surgical skill evaluation by force data for endoscopic sinus surgery training system in INTERNATIONAL CONFERENCE ON MEDICAL IMAGE COMPUTING AND COMPUTER-ASSISTED INTERVENTION, 2002. Springer), and tissue damage, motion repeatability, and path following (see, e.g., Hernandez, J., et al., SURGICAL ENDOSCOPY AND OTHER INTERVENTIONAL TECHNIQUES, 2004, 18(3): pp. 372-78; Moorthy, K., et al., BMJ, 2003, 327(7422): pp. 1032-37; Judkins, T.N., et al., J. OF ROBOTIC SURGERY, 2008. 1(4): pp. 307-12; Van Sickle, K.R., et al., SURGICAL ENDOSCOPY AND OTHER INTERVENTIONAL TECHNIQUES, 2005, 19(9): pp. 1227-31; Acosta, E. and B. Temkin, STUDIES IN HEALTH TECHNOLOGY AND INFORMATICS, 2005, 111: pp. 8-11). Most systems also use temporal metrics, such as completion time or time spent in various areas of the surgical workspace (Rosen, J., et al., COMPUTER AIDED SURGERY, 2002, 7(1): pp. 49-61; Cao et al., Task and motion analyses in endoscopic surgery in PROCEEDINGS ASME DYNAMIC SYSTEMS AND CONTROL DIVISION, 1996; Padoy, N., et al., A boosted segmentation method for surgical workflow analysis in INTERNATIONAL CONFERENCE ON MEDICAL IMAGE COMPUTING AND COMPUTER-ASSISTED INTERVENTION, 2007).

Motion analysis using descriptive statistics shows promise as a positive step for surgical skill assessment. Information about the task performance is presented to the user quickly and with reduced resources. However, evaluation systems also identify areas of difficulty during a task to improve future performance of the surgeon (see, e.g., Reiley, C.E., et al., SURGICAL ENDOSCOPY, 2011, 25(2): pp. 356-66).

Other recent work in building models to analyze skill, from Imperial College, has applied hidden Markov Models (HMMs) to model motion trajectories and predict skill levels (see, e.g., Leong, J.J., et al. HMM assessment of quality of movement trajectory in laparoscopic surgery in INTERNATIONAL CONFERENCE ON MEDICAL IMAGE COMPUTING AND COMPUTER-ASSISTED INTERVENTION, 2006, Springer). Tool tip movements were tracked using a Polaris 6-DOF infrared tracker during a view rotation MIS task. The study showed that HMMs can be used to learn models of surgical motion trajectories for users of different skill ability. Statistical methods, such as expectation-maximization, were used to calculate the maximum likelihood of HMM parameters. They compared the HMM-EM assessed scores with the OSATS and found a high correlation. However, this work strongly suggests that skill is indicative of motions and forces.

The work of Nagy and colleagues attempts to recognize gestures using optical character recognition (OCR) and HMMs in a method called Programming by Demonstration. Mayer, H., et al. The Endo [PA] R system for minimally invasive robotic surgery, in 2004 IEEE/RSJ INTERNATIONAL CONFERENCE ON INTELLIGENT ROBOTS AND SYSTEMS, 2004, IEEE. Analyzing experts performing the task multiple times, the system calculates an optimal path from these demonstrations. They record position and orientation of the tool tip and 2-DOF bending force of the tool shaft. Interestingly, they draw upon techniques adopted from bioinformatics and perform sequence alignment to classify similar portions of motions. Speidel et al. proposed computer vision approach for tracking of instruments in minimally invasive surgery based on endoscopic image sequences. Speidel, S., et al. Tracking of instruments in minimally invasive surgery for surgical skill analysis, in INTERNATIONAL WORKSHOP ON MEDICAL IMAGING AND VIRTUAL REALITY, 2006. In their proposed approach, the instruments were not modified, and the tracking was tested on sequences acquired during a real intervention. The generated trajectory of the instruments provides information that can be further used for surgical gesture interpretation.

The University of Washington independently performed a hierarchical decomposition of tasks in which the results were very similar to Cao’s. Their vocabulary was action- and movement-based: release, idle, hold, orient, pull/retract, push/reach, spread/grasp, translate/sweet as the eight subtasks. Rosen, J., et al., COMPUTER AIDED SURGERY, 2002, 7(1): p. 49-61.

Another automated movement recognition approach is a hybrid HMM-Support Vector Machine (SVM) model to segment a tele-operation peg-in-hole task online and offline. Castellani, A., et al. Hybrid HMM/SVM model for the analysis and segmentation of teleoperation tasks, IN IEEE INTERNATIONAL CONFERENCE ON ROBOTICS AND AUTOMATION, 2004, Proceedings, 2004. The hybrid classifier was used to segment a peg-in-hole task using force and torque data into four states.

Imperial College Surgical Assessment Device (ICSAD) utilizes an alternating current electromagnetic system with passive receivers attached to the dorsum of the hand over the mid-shaft of the third metacarpal. Bann, S.D., et al., WORLD J. OF SURGERY, 2003, 27(4): pp. 390-94. As hand movement takes place, a current is induced in the trackers, which is analyzed to determine the position of the hand/tracker. Data acquisition takes place at 20 Hz (rates of up to 100 Hz are possible). These raw positional data are analyzed by Bespoke software and calculate the number of movements, path length, and speed of movements for each hand. Noise is minimized by filtering the data. To calculate parameters, the number of movements for each hand are combined to give a total number of movements. For open surgery, in contrast to laparoscopic surgery, path length has been found to be nondiscriminatory; because this path length or distance traveled is used to calculate hand speed, it is not used for analysis.

Forestier et al. used decomposition of continuous kinematic data into a set of overlapping gestures represented by strings (bag of words) for which a comparative numerical statistic (tf-idf) can be computed, enabling the discriminative gesture discovery via its relative occurrence frequency. Forestier, G., et al., ARTIFICIAL INTELLIGENCE IN MED., 2018, 91: pp. 3-11. Their proposed approach, based on SAX-VSM algorithm, considers surgical motion as continuous multi-dimensional time-series and starts by discretizing them into sequence of letters (i.e., strings) using Symbolic Aggregate approXimation (SAX). In turn, SAX sequences are decomposed into subsequences of few consecutive letters via sliding window. The relative frequencies of these subsequences, i.e., the number of times they appear in a given sequence or in a set of sequences, are then used to identify discriminative patterns that characterize specific surgical motion. To discover the patterns, they rely on the vector space model (VSM) which has been originally proposed as an algebraic model for representing collection of text documents. The identified discriminative patterns are then used to perform classification by identifying them in to-be-classified recordings. Furthermore, by highlighting discriminative patterns in the visualization of original motion data, they are able to provide an intuitive visual explanation about why a specific skill assessment is provided.

In various implementations described herein, the measurement of the local information encoded in each video frame of a video of a surgical scene can be used to compute spatial and temporal entropy in multiple frames, which in turn can be used to predict bleeding in the surgical scene. Example systems identify and/or predict bleeding (e.g., arterial bleeding) based on the change in entropy of surgical scenes. Specific examples of techniques used to enhance bleeding prediction are described in Example 1. Example 1 also describes techniques for using entropy to predict bleeding. Example 2 reports the accuracy and robustness of an example technique for predicting bleeding from videos. This disclosure provides accurate and robust intelligent systems that assist and aid surgeons during minimally invasive surgery to prevent and control bleeding situations through the prediction of bleeding before an injury causing the bleeding occurs. Thus, these techniques can be used as a tool for preventing intraoperative bleeding altogether.

Various implementations of the present disclosure predict bleeding in a surgical field. Further, various implementations notify a surgeon of predicted bleeding within the surgical field. Accordingly, the surgeon may avoid surgical tool movements that would cause bleeding within the surgical field. In some cases, the surgical tool movements are automatically prevented. For instance, a surgical robot may dampen a movement of a surgical tool based on a prediction that the movement of the surgical tool will cause bleeding in the surgical field.

Various implementations described herein provide improvements to the technical field of surgical technology. For instance, implementations described herein can automatically and accurately predict bleeding within an intraoperative environment, and in some cases, prevent the surgeon from moving a surgical tool in such a way that will trigger the bleeding.

As used herein, the term “movement,” and its equivalents, can refer to a speed, a velocity, an acceleration, a jerk, or any higher-order differential of position.

As used herein, the term “local entropy,” and its equivalents, can refer to an amount of texture and/or randomness within a particular window of pixels. The “local entropy” of a pixel corresponds to the amount of texture and/or randomness in a window that includes the pixel.

As used herein, the term “white pixel,” and its equivalents, can refer to a pixel with one or more color channel values that exceed a particular threshold. For example, an RGB pixel may be a “white” pixel if the red channel component of the pixel exceeds a first threshold, the green channel component of the pixel exceeds a second threshold, and the blue channel component of the pixel exceeds a third threshold.

Implementations of the present disclosure will now be described with reference to the accompanying figures.

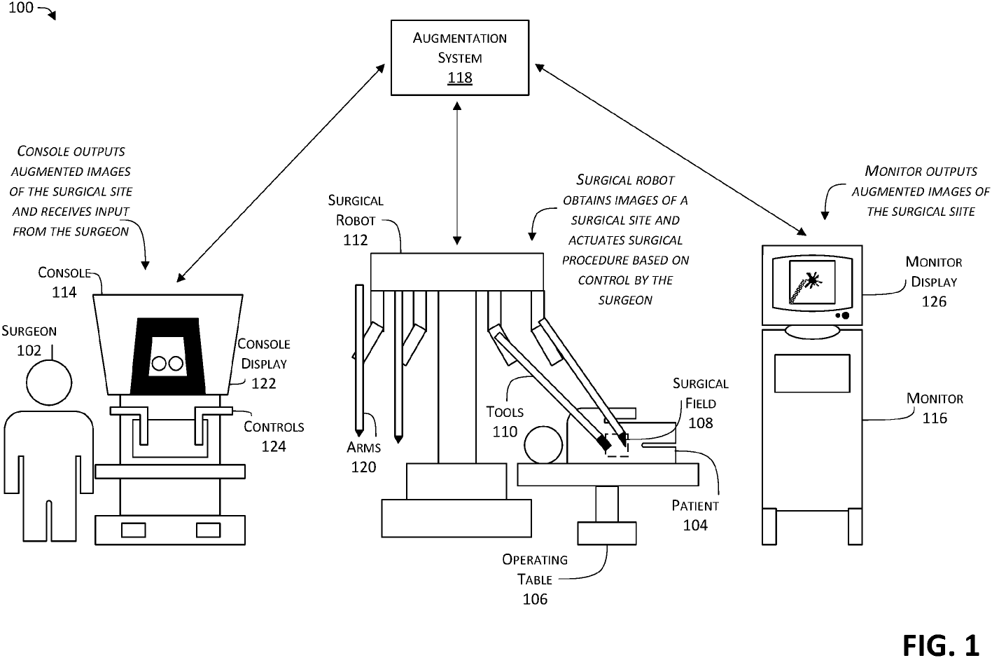

FIG. 1 illustrates an example environment 100 for predicting intraoperative bleeding. As illustrated, a surgeon 102 is operating on a patient 104 within the environment 100. In various cases, the patient 104 is disposed on an operating table 106.

The surgeon 102 operates within a surgical field 108 of the patient 104. The surgical field 108 includes a region within the body of the patient 104. In various cases, the surgeon 102 operates laparoscopically on the patient 104 using one or more tools 110. As used herein, the term “laparoscopic,” and its equivalents, can refer to any type of procedure wherein a scope (e.g., a camera) is inserted through an incision in the skin of the patient. The tools 110 include a scope, according to particular examples. In various cases, the tools 110 include another surgical instrument, such as scissors, dissectors, hooks, and the like, that is further inserted through the incision. The surgeon 102 uses the view provided by the scope to perform a surgical procedure with the surgical instrument on an internal structure within the surgical field 108 of the patient 104, without necessarily having a direct view of the surgical instrument. For example, the surgeon 102 uses the tools 110 to perform an appendectomy on the patient 104 through a small incision in the skin of the patient 104.

In various cases, the tools 110 include another surgical instrument, such as scissors, dissectors, hooks, and the like, that is further inserted through the incision. In various examples, the tools 110 include one or more sensors (e.g., accelerometers, thermometers, motion sensors, or the like) that facilitate movement of the tools 110 throughout the surgical field 108. In some implementations, the tools 110 include at least one camera and/or a 3-dimensional (3D) scanner (e.g., a contact scanner, a laser scanner, or the like) that can be used to identify the 3D positions of objects and/or structures within the surgical field 108. For example, images generated by the camera and/or volumetric data generated by the 3D scanner can be used to perform simultaneous localization and mapping (SLAM) or visual simultaneous localization and mapping (VSLAM) on the surgical field 108.

According to various implementations 102, the surgeon 102 carries out the procedure using a surgical system that includes a surgical robot 112, a console 114, a monitor 116, and an augmentation system 118. The surgical robot 112, the console 114, the monitor 116, and the augmentation system 118 are in communication with each other. For instance, the surgical robot 112, the console 114, the monitor 116, and the augmentation system 118 exchange data via one or more wireless (e.g., Bluetooth, WiFi, UWB, IEEE, 3GPP, or the like) interfaces and/or one or more wired (e.g., electrical, optical, or the like) interfaces.

In various examples, the surgical robot 112 may include the tools 110. The tools 110 are mounted on robotic arms 120. For instance, a first arm is attached to a scope among the tools 110, a second arm is attached to another surgical instrument, and so on. By manipulating the movement and location of the tools 110 using the arms 120, the surgical robot 112 is configured to actuate a surgical procedure on the patient 104. Although FIG. 1 is described with reference to the surgical robot 112, in some cases, similar techniques can be performed with respect to open surgeries, laparoscopic surgeries, and the like.

The console 114 is configured to output images of the surgical field 108 to the surgeon 102. The console 114 is includes a console display 122 that is configured to output images (e.g., in the form of video) of the surgical field 108 that are based on image data captured by the scope within the surgical field 108. In various examples, the console display 122 is a 3D display including at least two screens viewed by respective eyes of the surgeon 102. In some cases, the console display 122 is a two-dimensional (2D) display that is viewed by the surgeon 102.

The console 114 is further configured to control the surgical robot 112 in accordance with user input from the surgeon 102. The console 114 includes controls 124 that generate input data in response to physical manipulation by the surgeon 102. The controls 124 include one or more arms that are configured to be grasped and moved by the surgeon 102. The controls 124 also include, in some cases, one or more pedals that can be physically manipulated by feet of the surgeon 102, who may be sitting during the surgery. In various cases, the controls 124 can include any input device known in the art.

The monitor 116 is configured to output images of the surgical field 108 to the surgeon 102 and/or other individuals in the environment 100. The monitor 116 includes a monitor display 126 that displays images of the surgical field 108. In various examples, the monitor 116 is viewed by the surgeon 102 as well as others (e.g., other physicians, nurses, physician assistants, and the like) within the environment 100. The monitor display 126 includes, for instance, a two-dimensional display screen. In some cases, the monitor 116 includes further output devices configured to output health-relevant information of the patient 104. For example, the monitor 116 outputs a blood pressure of the patient 104, a pulse rate of the patient 104, a pulse oximetry reading of the patient 104, a respiration rate of the patient 104, or a combination thereof.

In various implementations of the present disclosure, the augmentation system 118 is configured to predict bleeding in the surgical field 108, cause details about the predicted bleeding to be indicated to the surgeon 102, dampen movements of the tools 110 to prevent the bleeding, or a combination thereof. In various examples, the augmentation system 118 is embodied in one or more computing systems. In some cases, the augmentation system 118 is located in the operating room with the surgical robot 112, the console 114, and the monitor 116. In some implementations, the augmentation system 118 is located remotely from the operating room. According to some examples, the augmentation system 118 is embodied in at least one of the surgical robot 112, the console 114, or the monitor 116. In certain instances, the augmentation system 118 is embodied in at least one computing system that is separated, but in communication with, at least one of the surgical robot 112, the console 114, or the monitor 116.

In various implementations, the augmentation system 118 receives image data from the surgical robot 112. The image data is obtained, for instance, by a scope among the tools 110. The image data includes multiple frames depicting the surgical field 108. According to various implementations, the multiple frames are at least a portion of a video depicting the surgical field 108. As used herein, the terms “image,” “frame,” and their equivalents, can refer to an array of discrete pixels. Each pixel, for instance, represents a discrete area (or, in the case of a 3D image, a volume) of an image. Each pixel includes, in various cases, a value including one or more numbers indicating a color saturation and/or grayscale level of the discrete area or volume. In some cases, an image may be represented by multiple color channels (e.g., an RGB image with three color channels), wherein each pixel is defined according to multiple numbers respectively corresponding to the multiple color channels. In some cases, the tools 110 include a 3D scanner that obtains a volumetric image of the surgical field 108.

The augmentation system 118 determines whether a movement of any of the tools is likely to cause bleeding by analyzing multiple frames in the image data. In some cases, the augmentation system 118 compares first and second frames in the image data. The first and second frames may be consecutive frames within the image data, or nonconsecutive frames. In some cases in which the first and second frames are nonconsecutive, and the augmentation system 118 repeatedly assesses the presence of bleeding on multiple sets of first and second frames in the image data, the overall processing load on the augmentation system 118 may be less than if the sets of first and second frames are each consecutive. In some implementations, the augmentation system 118 filters or otherwise processes the first and second frames in the image data.

According to particular implementations, the augmentation system 118 applies an entropy kernel (also referred to as an “entropy filter”) to the first frame and to the second frame. By applying the entropy kernel, the local entropy of each pixel within each frame can be identified with respect to a local detection window. In some implementations, an example pixel in the first frame or the second frame is determined to be a “low entropy pixel” if the entropy of that pixel with respect to its local detection window is under a first threshold. In some cases, an example pixel in the first frame or the second frame is determined to be a “high entropy pixel” if the entropy of that pixel with respect to its local detection window is greater than or equal to the first threshold. According to various implementations of the present disclosure, each pixel in the first frame and each pixel in the second frame is categorized as a high entropy pixel or a low entropy pixel.

The augmentation system 118 generates a first entropy mask based on the first frame and a second entropy mask based on the second frame. The first entropy mask can be a binary image with the same spatial dimensions as the first frame, wherein each pixel in the first entropy mask respectively corresponds to the categorization of a corresponding pixel in the first frame as a high entropy pixel or a low entropy pixel. For instance, an example pixel in the first entropy mask has a first value (e.g., 1 or 0) if the corresponding pixel in the first frame is a low entropy pixel or has a second value (e.g., 0 or 1) if the corresponding pixel in the first frame is a high entropy pixel. Similarly, the second entropy mask is a binary image with the same spatial dimensions as the second frame, wherein each pixel in the second entropy mask respectively corresponds to the categorization of a corresponding pixel in the second frame as a high entropy pixel or a low entropy pixel.

The augmentation system 118 predicts bleeding based on the first entropy mask and the second entropy mask, according to some implementations. According to various implementations, the augmentation system 118 generates a first masked image based on the first entropy mask and the first frame. For example, the first masked image includes at least some of the low-entropy pixels of the first frame. The low-entropy pixels correspond to pixels depicting homogenous elements of the frame, such as tools or blood. In some cases, the first masked image includes one or more color channels (e.g., the red color channel, the green color channel, the blue color channel, or a combination thereof) of the subset of pixels in the first frame with relatively low entropies. In some cases, the first masked image is generated by performing pixel-by-pixel multiplication of the first frame (or a single color channel of the first frame) with the first entropy mask, wherein the high-entropy pixels correspond to values of “0” and the low-entropy pixels correspond to values of “1” in the first entropy mask. The augmentation system 118 generates a second masked image based on the second entropy mask and the second frame, similarly to how the first masked image was generated.

In particular examples, the augmentation system 118 identifies a first pixel ratio (or number) corresponding to the number of “tool” pixels in the first masked image and identifies a second pixel ratio (or number) corresponding to the number of tool pixels in the second masked image. The tool pixels can refer to pixels with one or more color channel values that exceed one or more thresholds. In some cases, a pixel is determined to depict a tool if the red channel value of the pixel exceeds a first threshold, the green channel value of the pixel exceeds a second threshold, and the blue channel value of the pixel exceeds a third channel. For example, among the low-entropy pixels in the first frame, the pixels with relatively high color channel values are “white” pixels that correspond to tool 110 movement and/or position within the first frame. Similarly, among the low-entropy pixels in the second frame, the pixels with relatively high color channel values are “white” pixels that correspond to tool 110 movement and/or position within the second frame.

The augmentation system 118 identifies tool 110 movement within the first and second frames by comparing the first pixel ratio and the second pixel ratio. If the difference between the first pixel ratio and the second pixel ratio is less than a second threshold (e.g., 30%), then the augmentation system 118 concludes that the velocity of the tool 110 is unlikely to cause bleeding. However, if the difference between the first pixel ratio and the second pixel ratio is greater than or equal to the second threshold, then the augmentation system 118 predicts bleeding in the surgical field 108.

In some cases, the augmentation system 118 predicts bleeding based on an acceleration and/or jerk of the tool 110 in the surgical field. For instance, the augmentation system 118 can identify at least three masked images corresponding to at least three frames of a video of the surgical field 108. If the change in tool pixels between the at least three masked images indicates that the tool 110 is accelerating greater than a threshold amount, or a jerk of the tool 110 is greater than a threshold amount, then the augmentation system 118 predicts bleeding due to movement of the tool 110.

According to various implementations, the augmentation system 118 predicts bleeding based on kinematic data of the surgical robot 112. As used herein, the term “kinematic data” can refer to any combination of user input data, control data, and sensor data indicating position and/or movement of a surgical tool and/or a robotic arm. In various examples, the tools 110 include one or more sensors (e.g., accelerometers, thermometers, motion sensors, or the like) that facilitate movement of the tools 110 throughout the surgical field 108. In the context of FIG. 1, the console 114 generates user input data based on a manipulation of the controls 124 by the surgeon 102. The user input data may correspond to a directed movement of a particular tool 110 of the surgical robot 112 by the surgeon 102. In various examples, the augmentation system 118 receives the user input data and causes the surgical robot 112 to move the arms 120 and/or the tool 110 based on the user input data. For instance, the augmentation system 118 generates control data and provides (e.g., transmits) the control data to the surgical robot 112. Based on the control data, the surgical robot 112 moves or otherwise manipulates the arms 120 and/or the tool 110. In various cases, a sensor included in the particular tool 110 generates sensor data based on the movement and/or surrounding condition of the particular tool 110. The surgical robot 112 provides (e.g., transmits) the sensor data back to the augmentation system 118. The augmentation system 118, in some cases, uses the sensor data as feedback for generating the control data, to ensure that the movement of the particular tool 110 is controlled in accordance with the user input data. In various cases, the augmentation system 118 receives the user input data and the sensor data and generates the control data based on the user input data and the sensor data in a continuous (e.g., at greater than a threshold sampling rate) feedback loop in order to control the surgical robot 112 in real-time based on ongoing direction by the surgeon 102.

In some implementations, the augmentation system 118 identifies a velocity, an acceleration, a jerk, or some other higher order movement of the particular tool 110 based on the kinematic data. If the movement (e.g., the velocity, the acceleration, the jerk, or a combination thereof) is greater than a particular threshold, then the augmentation system 118 predicts that the movement is likely to cause bleeding in the surgical field 108.

In some cases, the augmentation system 118 can distinguish between different types of tools, and may selectively predict bleeding based on dangerous movements of tools that are configured to pierce tissue. For example, the augmentation system 118 may identify that the particular tool 110 is a scalpel, scissors, or some other type of tool configured to pierce tissue. The augmentation system 118 can predict that dangerous movements of the particular tool 110 will cause bleeding. However, another tool 110 that the augmentation identifies as being unable to pierce tissue will not be predicted as causing bleeding, even if it is identified as moving dangerously.

In some cases, the augmentation system 118 can track physiological structures (e.g., arteries, muscles, bones, tendons, veins, nerves, etc.) within the surgical field 108. According to some examples, the augmentation system 118 can use a combination of SLAM/VSLAM, image processing, and/or image recognition to identify what type of tissues are encountered by the tools 110 within the surgical scene. For instance, the augmentation system 118 can determine that the tool 110 is moving into an artery and is likely to cause bleeding. In some cases in which the augmentation system 118 determines that the tool 110 is encountering bone, the augmentation system 118 may refrain from predicting that the tool 110 will cause bleeding, even if the tool 110 is moving dangerously.

Using various techniques described herein, the augmentation system 118 can predict bleeding in the surgical field 108 before it occurs. Accordingly, the augmentation system 118 can indirectly prevent the bleeding by enabling the surgeon 102 to prevent the dangerous movement of the particular tool 110 before it causes the bleeding in the surgical field 108. For instance, the augmentation system 110 causes the console display 122 and/or the monitor display 126 to output the second frame. If the augmentation system 110 predicts bleeding, then the augmentation system 110 also causes the console 114 and/or the monitor 114 to output at least one augmentation indicating the predicted bleeding.

In some examples, the augmentation includes a visual augmentation. For instance, the augmentation system 118 causes the console display 122 and/or the monitor display 126 to output the second frame and a visual overlay that indicates the presence, location, and/or magnitude of the predicted bleeding. In particular examples, the visual overlay is a shape with a size and/or color that indicates the magnitude of the predicted bleeding. In some cases, the visual overlay is located (e.g., overlaid in) in a section of the second frame that depicts the predicted source of the bleeding. In some cases, the visual overlay is output in a location that is in a different portion of the second frame. In some cases, the visual overlay includes numbers and/or words indicating the presence, location, and/or magnitude of the predicted bleeding and/or dangerous tool movement. In some cases, the visual overlay indicates what physiological structure (e.g., which arteries, veins, etc.) is predicted to bleed due to the dangerous movement.

According to some cases, the augmentation includes a haptic augmentation. For example, the augmentation system 118 causes the controls 124 (e.g., joysticks, handles, and/or the pedals) to vibrate based on (e.g., simultaneously as) the predicted bleeding and/or dangerous tool movement.

In some instances, the augmentation includes an audio augmentation. For instance, the augmentation system 118 causes at least one speaker among the console 114 or the monitor 116 to output a sound indicating the predicted bleeding and/or dangerous tool movement. In various implementations described herein, any output capable of indicating, to the surgeon 102, that the occurrence, the location, and/or the magnitude of predicted bleeding and/or dangerous tool movement can be triggered by the augmentation system 118.

In some cases, the augmentation system 118 directly prevents the bleeding by dampening the dangerous movement of the particular tool 110. For instance, the augmentation system 118 adjusts the control data to prevent the particular tool 110 to continue the dangerous tool movement, even if the user input data indicates that the surgeon 102 has continued to direct the dangerous tool movement. In some cases, the augmentation system 118 generates the control data to lower the velocity, acceleration, jerk, or other dangerous movement otherwise directed by the user input data.

In various implementations described herein, the augmentation system 118 predicts bleeding in the surgical field 108 due to dangerous tool movement. The bleeding can be predicted based on spatio-temporal entropy and/or kinematic data. In some examples, the augmentation system 118 further indicates predicted bleeding and/or dangerous tool movement to the surgeon 102. According to some cases, the augmentation system 118 can automatically dampen the dangerous tool movement, thereby preventing the bleeding from occurring.

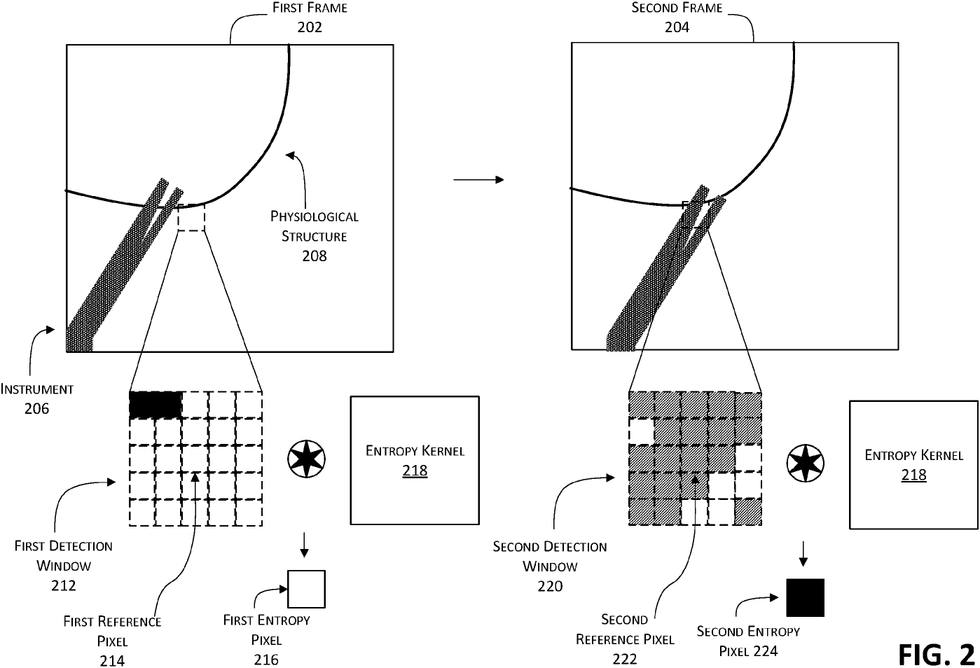

FIG. 2 illustrates example techniques for generating entropy pixels representing the entropy of pixels in frames depicting a scene of interest. In various cases, the entropy pixels are generated by an augmentation system, such as the augmentation system 118 described above with reference to FIG. 1.

In the example illustrated in FIG. 2, a first frame 202 depicts a scene of interest (e.g., the surgical field 108 described above with reference to FIG. 1) at a first time and a second frame 204 depicts the scene of interest at a second time. The second time is subsequent to the first time. In various instances, the first frame 202 and the second frame 204 are obtained with by the same imaging device, such as the same scope (e.g., a laparoscope, an endoscope, or some other camera). According to some examples, the first frame 202 and the second frame 204 are consecutive images, such that a difference between the first time and the second time is a sampling period of the imaging device. In some cases, the first frame 202 and the second frame 204 represent images, in which the difference between the first time and the second time is greater than the sampling period of the imaging device. For instance, the first frame 202 and the second frame 204 are nonconsecutive frames in a video. In some cases, more than two frames in the video can be analyzed.

In FIG. 2, the first frame 202 and the second frame 204 are two-dimensional images, but implementations are not limited thereto. In some cases, the first frame and the second frame 204 are represented by arrays of pixels. Each pixel is defined according to an area (e.g., a square area) and at least one value. In some examples in which the first frame 202 and the second frame 204 are color images, a value of a pixel is defined according to three numbers (e.g., each being in a range of 0 to 255, inclusive) corresponding to red, green, and blue (RGB) components, or cyan, magenta, yellow (CMY) components, of the color of the area defined by the pixel. In some examples in which the first frame 202 and the second frame 204 are binary images, a value of a pixel is defined as 0 (e.g., white or non-red) or 1 (e.g., black). In some examples in which the first frame 202 and the second frame 204 are grayscale images, a value of a pixel is defined according to a single number in a range (e.g., of 0 to 255, inclusive) representing a gray value of the area defined by the pixel. However, implementations are not limited to the specific color models described herein. In some cases, the first frame 202 and the second frame 204 represent a single color channel, such as the red component of image data obtained from the imaging device.

As shown, the first frame 202 depicts an instrument 206 and a physiological structure 208 within the scene of interest. However, the instrument 206 has moved between the first time and the second time. In some cases, the movement of the instrument 206 represents a dangerous movement likely to cause bleeding at a third time subsequent to the second time.

To identify the dangerous movement, entropy maps including entropy pixels are generated based on the first frame 202 and the second frame 204. For instance, a first detection window 212 is defined as a square portion of pixels in the first frame 202. Although the first detection window 212 is depicted as having dimensions of 5×5 pixels in FIG. 2, implementations are not limited thereto. For example, the first detection window 212 can have dimensions of 9×9 pixels, 11×11 pixels, or the like. The first detection window 212 includes a first reference pixel 214. In some cases, the first reference pixel 214 is located in the center of the first detection window 212.

In various cases described herein, bleeding can be predicted in a frame by measuring the uniformity of different regions of the frame. The uniformity of the different regions can be determined based on the concept on entropy. For instance, an entropy filter can be used to produce a texture distribution of a frame. A morphological methodology using the entropy filter can be used to extract salient motions or objects that appear to be moving within an entropy mapped frame. The entropy of the frame can be representative of a variational statistical measurement of the frame. The morphological methodology can have more robustness in relation to noise compared to traditional difference-based methods. See Jaiswal, J. OF GLOBAL RESEARCH IN COMPUTER SCI., 2011, 2(6): pp. 35-38. The detection accuracy of the morphological methodology can be improved by using the entropy from multiple frames in a video.

According to various implementations, a series of processing steps can be performed in order to predict bleeding by detecting unsafe tool movement within one or more frames of a video. First, a frame depicting a surgical scene can be generated (e.g., the frame may be part of a video depicting the surgical scene) and can be converted from the RGB color model to the grayscale color model to eliminate hue and saturation components, but to retain a luminance component of the first frame. A moving, two-dimensional k by k window (wherein k is an integer number of pixels) may be applied to the grayscale image, and the local entropy of the image in the window is computed to generate a grayscale entropy map of that frame. An entropy mask can be generated by binarizing the entropy map. Pixels corresponding to lower than a threshold entropy (i.e., “low-entropy pixels”) are defined by one value (e.g., “1” or “0,” “black” or “white”) and the other pixels (i.e., “high-entropy pixels”) are defined by another value (e.g., “0” or “1,” “white” or “black). The total number of low-entropy pixels in the entropy map of the frame can be determined and compared to that of a previous frame. If the change is greater than a threshold (e.g., a pre-set threshold), the original frame can be labeled as “dynamic.” Temporal change in the entropy masks is a basis for detecting unsafe tool movement. An abrupt increase in the number of low-entropy pixels whose color component(s) are greater than one or more thresholds (also referred to as “white” pixels) in the first masked RGB frame and the second masked RGB frame can be correlated to regions of tool movement in the dynamic image sequence.

Local entropy can be used to quantify and represent homogeneity of a small area in a frame. More specifically, for a square region of size k by k, the local entropy can be calculated according to the following Equation 1:

$$L E \left({Ψ}_{r}\right) = {\sum }_{i = 0}^{k} {\sum }_{j = 0}^{k} {p}_{i j} l o {g}_{2} \left({p}_{i j}\right) \qquad \text{Eq. 1}$$

where pᵢⱼ represents the probability function for a pixel [i,j]. The entropy map is represented as a grayscale image with higher intensities for regions that are less homogenous (regions that correspond to areas of higher entropy and/or information) and lower intensity for the regions they are more homogenous (regions that correspond to areas of lower entropy and/or information).

Local entropy values can be used to evaluate the gray-level spread in the histogram. A local entropy of a window is associated with the variance exhibited by pixels in the window and represents textural features of the pixels in the window. Computing the local entropy of each pixel in a frame can be used to generate an entropy map of the frame. The generated entropy map can be a grayscale image which maps the different regions of the frame with different amounts of homogeneity. In the context of a frame depicting a surgical tool, the frame can be associated with lower local entropy values in regions depicting the tool than in regions depicting tissue or other structures. This is because the areas depicting the tool are more homogenous (e.g., uniform) due to the smooth image texture of the tool.

In some cases, bleeding can be predicted in a video depicting a robotic surgery using the concept of entropy and homogeneity. In some examples, the entropy map of each frame in the video can be generated by calculating the local entropy of each pixel in each frame. The entropy maps may be represented as grayscale images. The entropy maps may be binarized, such that areas corresponding to relatively high entropy levels are set at a first value and areas corresponding to relatively low entropy levels are set at a second value. The frames can be masked with their respective entropy maps. The change of randomness/homogeneity in consecutive frames over time can be calculated based on the masked frames. Hence, LE(Ψᵣ) and pᵢⱼ can be functions of time. For clarity, these metrics can be expressed as LE(Ψ_{r,n}) and pᵢⱼ (n), respectively, where n means the n-th frame in the video.

The change in intensity due to tool movement can be quantified through rate of local change of uniformity that is formulated in accordance with Equation 2:

$$\text{R} L E \left({Ψ}_{r}\right) = \frac{L H \left({Ψ}_{r} , n\right) - L H \left({Ψ}_{r} , n - 1\right)}{L H \left({Ψ}_{r} , n - 1\right)} \qquad \text{Eq. 2}$$

where RLE is the relative local entropy of region Ψᵣ. Equation 2 can be used to quantify two characteristics of a video: the rate of change in homogeneity of frames within the video through the value of RLE(Ψᵣ) and the coordinates i,j of the changes, which can be interpreted as the spatial homogeneity within the image. This means that by setting the value of RLE(Ψᵣ) to be less than a certain value, a threshold of the ratio of white pixels, it is possible to detect bleeding, and the i and j values can be used to locate and outline the region of interest, which may correspond to the origin of a predicted bleeding area.

For real-time prediction of bleeding, changes in the distributions of entropy maps corresponding to consecutive frames can be tracked as the frames are obtained (e.g., in a video stream). That is, the entropy map from each frame can be compared to the entropy map of the previous frame over a time period. Each entropy map can localize regions with a high degree of homogeneity. For the sake of quantification, the entropy maps can be binarized into entropy masks, and the number of low-entropy pixels (e.g., the total number of pixels corresponding to less than a threshold entropy) can be calculated as an indicator of uniformity of different regions of the content of the video with respect to time.

To identify bleeding regions, an entropy map can be divided into two types of regions: homogeneous regions and heterogenous regions. The entropy map may be binarized into an entropy mask, which can allow for the identification of uniform regions within the video frame with low intensity, and heterogenous (texturized) regions with the highest intensity. When a current RGB frame is masked by its binarized entropy mask, a masked-RGB frame is produced, wherein pixels corresponding to the heterogenous regions are removed and RGB pixels corresponding to the homogenous regions (corresponding to pixels depicting the tool) are retained. The pixels corresponding to the homogenous regions are also referred to as “color” pixels. Some of the color pixels may include what are referred to herein as “white” pixels.

The white pixels within the masked RGB frame indicate the homogeneity within an image introduced by one or more tools depicted in the RGB frame. Measuring the number of white pixels (e.g., pixels whose red channel, green channel, and blue channel values are above certain thresholds) in a masked frame, and the variation of the numbers of red pixels in multiple successive masked frames, allows for detection of bleeding frames as well as localization of the bloody spots. The color pixels can also include “red” pixels (e.g., pixels whose red channel values are above a certain threshold, but whose blue and/or green channel values are less than particular thresholds), which may correspond to bleeding or other, non-tool homogenous structures within the frame.

The thresholds and rates of change of the entropy can be identified by computing the rate of change between white pixels for two consecutive masked-RGB frames. Comparing the raw temporal entropies of two successive frames may lead to high sensitivity to small changes in local uniformity, causing large fluctuations and poor robustness. Lack of robustness will, in turn, lead to false prediction of bleeding. To improve robustness, a moving average low-pass filter can be applied to the masked frames to smooth the changes in entropy for one or more previous frames preceding the current frame. The threshold for predicting bleeding when computing the temporal entropy can be represented by the following Equation 3, and may be proportional to the ratio of the image size to the size of the neighborhood (k by k) that is used for generating the entropy map:

$$T {E}_{T h} ∝ \frac{w \times h}{L {E}_{w} \times L {E}_{h}} \qquad \text{Eq. 3}$$

This threshold can be computed by introducing the coefficient α, which is an empirically derived value. The following Equation 4 can be used to calculate the threshold based on α.

$$T {E}_{T h} = a \times \frac{w \times h}{{A}_{L}} \qquad \text{Eq. 4}$$

Here, w is the width of input image, h is the height of the image, and A_{L} is the window area used for computing the local entropy. α is the empirical coefficient. For example, α can be empirically derived based on training videos sets. Adjusting the value of α can impact the sensitivity and/or specificity of the method. Thus, the value of α can be set in order to achieve a particular sensitivity and/or specificity when the method is tested with respect to the training video sets. In some experimental samples, setting the value of α equal to 0.01 achieved acceptable results in terms of sensitivity and specificity.

As previously mentioned, the prediction of arterial bleeding is based on the change and number of white pixels within the masked RGB frame. Setting the appropriate threshold for counting the number of white pixels for a certain interval can play a critical role in avoiding false prediction of bleeding. This threshold is based on the following Equation 5:

$$\begin{matrix}\text{for p} ∈ {\text{M}}^{\left(\text{w} \times \text{h}\right)} , \text{if} \left({\text{P}}_{\text{R}}\right) \overline{\text{-}} {σ}_{\text{R}} < {\text{p}}_{\text{R}} < \left({\text{P}}_{\text{R}}\right) \overline{+} {σ}_{\text{R}} , \\ \text{then p is a red pixel}\end{matrix} \qquad \text{Eq. 5}$$

where p is any random pixel and belongs to the masked RGB frame M with a size of w×h, (Pᵣ) ̅is the mean of the pixels’ red channel intensities of the masked RGB frame, and σ_{R} is the standard deviation.

Referring back to FIG. 2, a first entropy pixel 216 is generated by calculating the entropy within the first detection window 212 with respect to the first reference pixel 214. In various cases, the first entropy pixel 216 is generated by applying (e.g., convolving or cross-correlating) an entropy kernel 218 with the first detection window 212. For example, a value of the first entropy pixel 216 is based on an output of a convolution operation of a matrix representing the values of the pixels in the first detection window 212 with a matrix defining the entropy kernel 218. In some cases, the value of the first entropy pixel 216 is based on a Shannon entropy of the first detection window 212. In various cases, the value of the first entropy pixel 216 is based on a local entropy with respect to the first reference pixel 214.

In some examples, the value of the first entropy pixel 216 is binarized. For instance, if the entropy of the first detection window 212 is greater than or equal to a first threshold, then the first entropy pixel 216 is assigned a first value. If the entropy of the first detection window 212 is less than the first threshold, then the first entropy pixel 216 is assigned a second value. In the example illustrated in FIG. 2, the first entropy pixel 216 is assigned the first value, indicating that the first reference pixel 214 is a high-entropy pixel in the first frame 202.

According to various implementations, a first entropy mask including binarized multiple entropy pixels (including the first entropy pixel 216) is generated based on the first frame 202. The first entropy mask is a binary image, wherein each pixel of the first entropy mask indicates an entropy associated with a corresponding pixel in the first frame 202. The first frame 202 and the first entropy mask may have the same dimensions. In various cases, the first detection window 212 is a sliding window that can be used to generate the entropy of each pixel in the first frame 202.

Similarly, a second entropy mask is generated for the second frame 204. As illustrated in FIG. 2, a second detection window 220 (similar to the first detection window) is used to determine the entropy associated with a second reference pixel 222 in the second frame 204. A second entropy pixel 224 is generated by applying the entropy kernel 218 to the second detection window 220. A value of the second entropy pixel 224 is the binarized output of the application of the entropy kernel 218 to the second detection window 220. In the example illustrated in FIG. 2, the entropy of the second reference pixel 222 is less than or equal to the first threshold, such that the second reference pixel 222 is a low-entropy pixel and the second entropy pixel 224 has the second value. In some cases, a second entropy mask representing the entropy of each pixel in the second frame 204 is generated.

The first and second entropy masks can be used to detect the dangerous movement of the instrument 206 between the first time and the second time. In various cases, an indication of the dangerous movement and/or predicted bleeding can be output to a user, such as a surgeon performing a procedure depicted in the first frame 202 and the second frame 204. In some cases, the movement of the instrument 206 can be dampened at the third time, thereby preventing the bleeding from occurring.

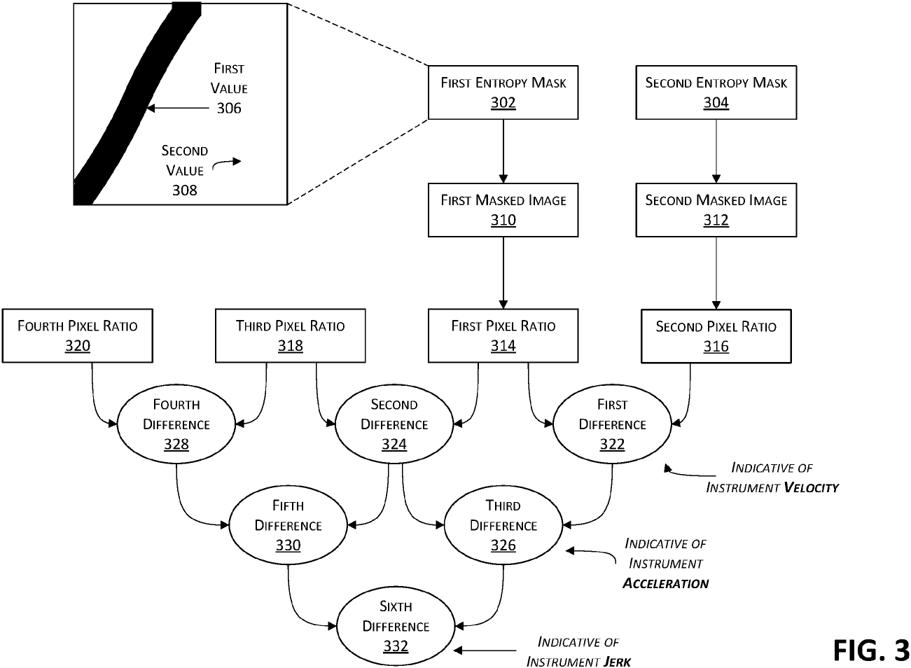

FIG. 3 illustrates an example of a technique for predicting bleeding based on entropy maps. Specifically, FIG. 3 illustrates a first entropy mask 302 and a second entropy mask 304. In some implementations, the first entropy mask 302 is generated based on the first frame 202 described above with reference to FIG. 2, and the second entropy mask 304 is generated based on the second frame 204 described above with reference to FIG. 2. In various cases, the first entropy mask 302 has the same pixel dimensions (e.g., number of columns and/or rows of pixels) as the first frame 202, and the second entropy mask 304 has the same pixel dimensions as the second frame 204. For example, the first entropy mask 302 includes the first entropy pixel 216 and the second entropy mask 304 includes the second entropy pixel 224. According to particular examples, the technique illustrated by FIG. 3 is performed by a system, such as the augmentation system 118 described above with reference to FIG. 1 and/or a separate computing system.

The first entropy mask 302 and the second entropy mask 304 are each binary images, according to various implementations. Some of the pixels in the first entropy mask 302 and the second entropy mask 304 have a first value 308. The first value 308 indicates pixels in the first frame 202 and the second frame 204 with calculated entropy values that are greater than or equal to the first threshold. Some of the pixels in the first entropy mask 302 and the second entropy mask 304 have a second value 306. The second value 306 indicates pixels in the first frame 202 and the second frame 204 with calculated entropy values that are less than a first threshold (e.g., the pixels are “low-entropy” pixels).

A first masked image 310 is generated based on the first entropy mask 302 and the first frame 202. In various examples, the first masked image 310 represents at least a subset of the pixels of the first frame 202 with entropies that are less than or equal to the first threshold. For instance, if the second value 306 is 1, the first masked image 310 is generated by performing pixel-by-pixel multiplication of the first entropy mask 302 and the first frame 202.

Similarly, a second masked image 312 is generated based on the second entropy mask 304 and the second frame 204. In various examples, the second masked image 312 represents at least a subset of the pixels of the second frame 204 with entropies that are less than or equal to the first threshold. For instance, if the second value 306 is 1, the second masked image 312 is generated by performing pixel-by-pixel multiplication of the second entropy mask 304 and the second frame 204 (e.g., the red channel of the second frame 204).

A first pixel ratio 314 is generated based on the first masked image 312. In various examples, the first pixel ratio 314 represents the number of low-entropy pixels (e.g., pixels with local entropies less than or equal to the first threshold) in the first masked image 310 with color values (e.g., red, green, and blue channel values) greater than at least one particular threshold, divided by the total number of pixels in the first masked image 310. Thus, the first pixel ratio 314 corresponds to the ratio of low-entropy white pixels in the first masked image 310. These low-entropy white pixels in the first masked image 310 correspond to an instrument (e.g., a surgical tool) depicted in the first frame 202 and are referred to as “tool” pixels.

Similarly, a second pixel ratio 316 is generated based on the second masked image 312. In various examples, the second pixel ratio 316 represents the number of low-entropy pixels in the second masked image 312 with color values that are greater than at least one particular threshold divided by the total number of pixels in the second masked image 312. Thus, the second pixel ratio 316 corresponds to the ratio of low-entropy white pixels in the second masked image 312. These low-entropy white pixels in the second masked image 312 correspond to the instrument (e.g., a surgical tool) depicted in the second frame 204, and are also tool pixels.

In various implementations, bleeding is predicted based on the first pixel ratio 314 and the second pixel ratio 316. In some cases, bleeding is predicted based on the number of tool pixels in the first masked image 310 and the number of tool pixels in the second masked image 312. In some implementations, a dangerous instrument movement is detected when the first pixel ratio 314 and the second pixel ratio 316 are sufficiently different. For instance, a first difference 322 between the first pixel ratio 314 and the second pixel ratio 316 is compared to a second threshold. In various cases, the first difference 322 relates to the increase in global entropy from the first frame 202 to the second frame 204. If the first difference 322 is less than the second threshold, then the movement of the instrument depicted in the first frame 202 and the second frame 204 is determined to be safe (e.g., not dangerous) and relatively unlikely to cause bleeding. However, if the first difference 322 is greater than or equal to the second threshold, then the movement of the instrument is determined to be dangerous and likely to cause bleeding. If the dangerous movement is detected, then the movement can be dampened, thereby preventing the movement from causing the bleeding at a time subsequent to when the second frame 204 is obtained.

Although FIG. 3 is described as comparing the tool pixels associated with two frames, the dangerous movement can also be detected based on tool pixels associated with more than two frames. The first difference 322 corresponds to a first derivative of the position of the instrument across the first frame 202 and the second frame 204. For instance, the first difference 322 is indicative of instrument velocity in the first frame 202 and the second frame 204, but the entropies of other (e.g., earlier) frames can be compared to identify the magnitude of higher-order movements of the instrument.

In various examples, a third pixel ratio 318 is generated based on a third frame that precedes the first frame 202. A second difference 324 between the third pixel ratio 218 and the first pixel ratio 314 is identified. In addition, a third difference 326 between the second difference 324 and the first difference 322 is also calculated. The third difference 326 corresponds to a second derivative of the position of the instrument depicted across the third frame, the first frame 202, and the second frame 204. For instance, the third difference 326 is indicative of instrument acceleration across the third frame, the first frame 202, and the second frame 204. In some examples, if the third difference 326 is less than a third threshold, then the movement of the instrument is determined to be safe and unlikely to cause bleeding. However, if the third difference 326 is greater than or equal to the third threshold, then the movement of the instrument is determined to be dangerous and likely to cause bleeding.

In some cases, a jerk of the instrument is assessed. A fourth pixel ratio 320 is generated based on a fourth frame that precedes the third frame. A fourth difference 328 between the fourth pixel ratio 320 and the third pixel ratio 318 is identified. A fifth difference 230 is generated between the fourth difference and the second difference 324. Further, a sixth difference 332 is generated between the fifth difference 330 and the third difference 326. The sixth difference 332 corresponds to a third derivative of the position of the instrument depicted across the fourth frame, the third frame, the first frame 202, and the second frame 204. For instance, the sixth difference 332 is indicative of instrument jerk across the fourth frame, the third frame, the first frame 202, and the second frame 204. In some examples, if the sixth difference 332 is less than a fourth threshold, then the movement of the instrument is determined to be safe and unlikely to cause bleeding. However, if the sixth difference 332 is greater than or equal to the fourth threshold, then the movement of the instrument is determined to be dangerous and likely to cause bleeding.

Thus, higher-order derivatives of the spatio-temporal entropy of the surgical scene containing the instrument can be evaluated in order to assess various movements of the instrument. In some cases, an acceleration over a particular threshold, a jerk over a particular threshold, or any other type of higher-order movement over a particular threshold, is indicative of a dangerous movement. Bleeding in the surgical scene is predicted based on the detection of the dangerous movement. If the dangerous movement is detected, then the movement can be dampened, thereby preventing the movement from causing the bleeding at a time subsequent to when the second frame 204 is obtained.

The various thresholds (e.g., the first threshold, the second threshold, the third threshold, the fourth threshold, or the like) are adjustable, in some implementations. For example, any of the first threshold, the second threshold, the third threshold, and/or the fourth threshold can be set at a relatively high level (e.g., 40% or 0.4) for surgical procedures that are particularly sensitive to intraoperative bleeding, such as neurological procedures. In contrast, any of the first threshold, the second threshold, the third threshold, and/or the fourth threshold can be set at a relatively high level (e.g., 10% or 0.1) for surgical procedures that are relatively insensitive to intraoperative bleeding, such as orthopedic procedures. In various cases, a surgeon or other user can input the sensitivity and/or any of the first threshold, the second threshold, the third threshold, and/or the fourth threshold into the system (e.g., the augmentation system 118) that is predicting bleeding in the surgical scene.

In some cases, upon detecting a dangerous movement, the system automatically dampens the movement of the instrument. For example, if a surgical robot is moving the instrument, then the system can output an instruction to slow, decelerate, or otherwise prevent the robot from continuing the dangerous movement of the instrument. Accordingly, the predicted bleeding can be automatically prevented.

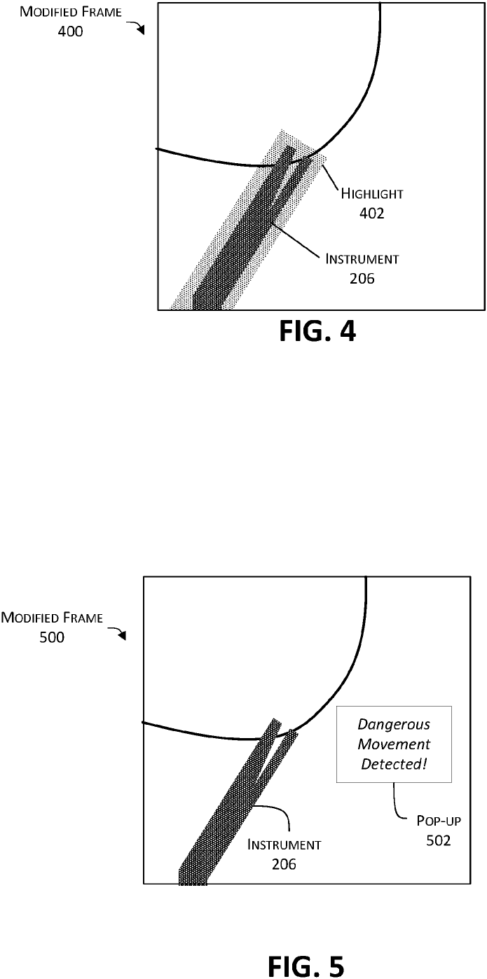

In particular examples, the system outputs, to a user (e.g., a surgeon), an indication of the dangerous movement. This indication may enable the user to refrain from continuing to direct the dangerous movement of the instrument, thereby preventing the user from causing the predicted bleeding in the surgical scene. The indication may be output in any manner that is discernable to the user, such as via audio feedback, haptic feedback, visual feedback, or the like. Some examples of visual indications of dangerous movements are described with reference to FIGS. 4 and 5.

FIG. 4 illustrates an example of a modified frame 400 indicating a dangerous movement by highlighting. In various cases, the modified frame 400 can include a frame of the surgical scene that is obtained when, or shortly after (e.g., the frame immediately subsequent to), a dangerous movement is identified. For instance, the modified frame 400 can include the second frame 204 described above with reference to FIG. 2. The modified frame 400 depicts the instrument 206. Further, the modified frame 400 includes a highlight 402. The highlight 402 may emphasize the instrument 206 depicted in the modified frame 400. For instance, the highlight 402 may represent a shape or a line disposed around an edge of the depicted instrument 206. In some cases, the highlight 402 may include a particular color (e.g., green, pink, or the like) that is otherwise missing from the modified frame 400. In some cases, the color of the highlight 402 depends on the severity of the dangerous movement. For instance, if the jerk of the instrument 206 is above a first threshold but not a second threshold, then the highlight 402 is yellow, but if the jerk of the instrument 206 is above both the first threshold and the second threshold, then the highlight 402 is orange. Accordingly, the presence of the highlight 402 is easily discernable to a viewer. The presence of the highlight 402 indicates that the instrument 206 is moving dangerously. Accordingly, upon viewing the highlight 402, the viewer (e.g., a surgeon) can adapt the movement of the instrument 206 to prevent bleeding.

FIG. 5 illustrates an example of a modified frame 500 indicating a dangerous movement by pop-up notifications. In various cases, the modified frame 500 can include a frame of the surgical scene that is obtained when, or shortly after (e.g., the frame immediately subsequent to), a dangerous movement is identified. For instance, the modified frame 500 can include the second frame 204 described above with reference to FIG. 2. The modified frame 500 depicts the instrument 206. Further, the modified frame 500 includes a pop-up 502. The pop-up 502 is overlaid on the frame depicting the surgical scene. The pop-up 502 includes, for instance, a message indicating that the movement of the instrument 206 is dangerous and/or likely to cause intraoperative bleeding. Accordingly, upon viewing the pop-up 502, the viewer (e.g., a surgeon) can adapt the movement of the instrument 206 to prevent bleeding. In some cases, the pop-up 502 further indicates a physiological structure likely to be injured by the dangerous movement. For example, the pop-up 502 could identify a particular vein or artery in the vicinity (e.g., within a threshold distance) of the instrument 206 when the dangerous movement is detected.

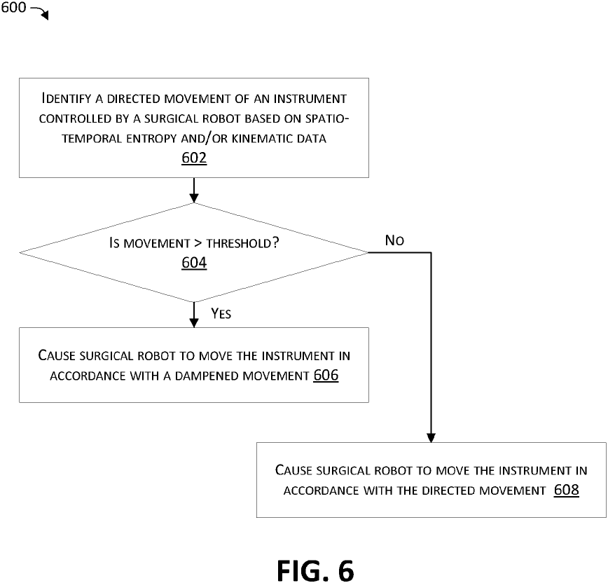

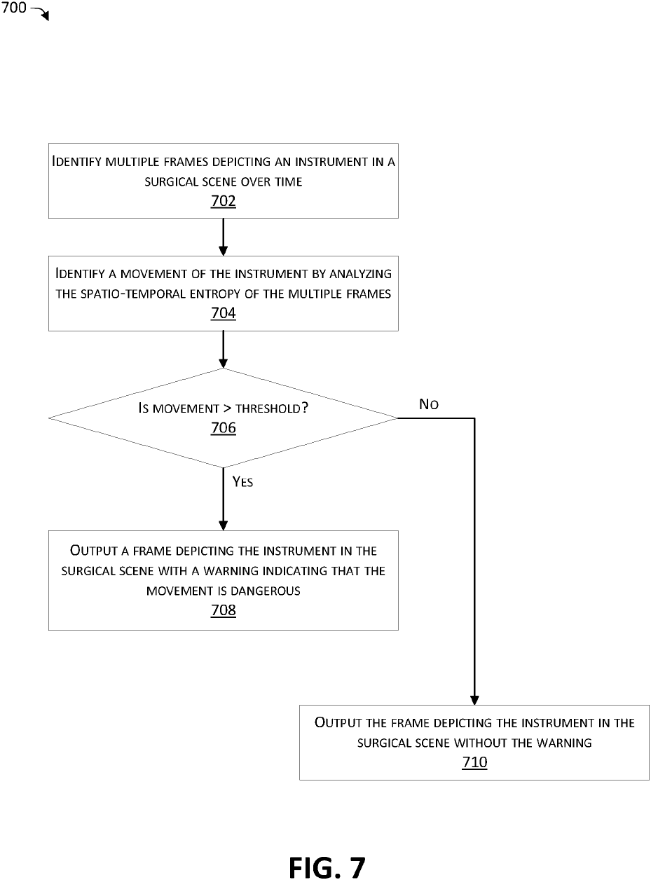

FIGS. 6 and 7 illustrate processes that can be performed by various devices, such as computing systems. In some cases, the processes illustrated in FIGS. 6 and 7 can be performed by a medical device, a surgical system, a surgical robot, or some other system (e.g., the augmentation system 118 described above with reference to FIG. 1). Unless otherwise specified, the steps illustrated in FIGS. 6 and 7 can be performed in different orders than those specifically illustrated.

FIG. 6 illustrates a process 600 for preventing intraoperative bleeding in a robotic surgical environment. At 602, the entity performing the process 600 identifies a directed movement of an instrument controlled by a surgical robot. The directed movement is identified based on spatio-temporal entropy and/or kinematic data associated with the movement of the instrument. In various implementations, the directed movement is based on a user input received by the entity. In some cases, the user input is by a surgeon utilizing the instrument in a surgical procedure. According to various implementations, the movement includes at least one of a velocity, an acceleration, or a jerk of the instrument. In some cases, the movement includes a higher-order derivative of a position of the instrument within a surgical scene.

In some cases, spatio-temporal entropy of multiple frames depicting the instrument are analyzed in order to identify the movement. For instance, masked images corresponding to multiple frames in a video of the instrument in the surgical scene are generated. A number and/or ratio of low-entropy white pixels (also referred to as tool pixels) within the masked images are compared. The low-entropy white pixels can be used to distinguish changes in entropy corresponding to tool movement, rather than other sources of entropy changes within the frames (e.g., bleeding, such as arterial bleeding). Based on the comparison, the movement of the instrument can be identified.

In various cases, kinematic data corresponding to a surgical robot that is directing the movement of the instrument is analyzed. The kinematic data may be based, at least partly, on the user input directing the movement of the instrument.

At 604, the entity determines whether the movement is greater than a threshold. In some cases, the threshold is assigned based on a user input. For instance, the surgeon may input a threshold that is relatively low for a surgical procedure that is relatively sensitive to sudden instrument movements (e.g., neurosurgical procedures). In some cases, the surgeon may input a threshold that is relatively high for a surgical procedure that is relatively insensitive to sudden instrument movements (e.g., orthopedic procedures). According to some examples, the surgeon can change the threshold for different stages of a single procedure. For instance, the surgeon may input a threshold that is relatively high during a suturing stage of the procedure, but may input a threshold that is relatively low during another stage of the procedure.

If the entity determines that the movement is greater than the threshold at 604, then the process 600 proceeds to 606. At 606, the entity causes the surgical robot to move the instrument in accordance with a dampened movement. The dampened movement, for instance, is dampened with respect to the directed movement. In some implementations, the dampened movement has at least one of a lower velocity, a lower acceleration, or a lower jerk than the directed movement.

If, on the other hand, the entity determines that the movement is not greater than the threshold at 604, then the process proceeds to 608. At 608, the entity causes the surgical robot to move the instrument in accordance with the directed movement. In some examples, process 600 can be performed repeatedly, thereby monitoring the surgical environment in real-time.

FIG. 7 illustrates a process 700 for preventing intraoperative bleeding in a surgical environment. At 702, the entity performing the process 700 identifies multiple frames depicting an instrument in a surgical scene over time. In various examples, the multiple frames are obtained from a scope capturing the frames. For instance, the multiple frames are at least a part of a video of the surgical scene.

At 704, the entity identifies a movement of the instrument by analyzing the spatio-temporal entropy of the multiple frames. For instance, masked images corresponding to multiple frames in a video of the instrument in the surgical scene are generated. A number and/or ratio of low-entropy white pixels within the masked images are compared. The low-entropy white pixels can be used to distinguish changes in entropy corresponding to tool movement, rather than other sources of entropy changes within the frames (e.g., bleeding, such as arterial bleeding). Based on the comparison, the movement of the instrument can be identified.

At 706, the entity determines whether the movement is greater than a threshold. In some cases, the threshold is assigned based on a user input. For instance, the surgeon may input a threshold that is relatively low for a surgical procedure that is relatively sensitive to sudden instrument movements (e.g., neurosurgical procedures). In some cases, the surgeon may input a threshold that is relatively high for a surgical procedure that is relatively insensitive to sudden instrument movements (e.g., orthopedic procedures). According to some examples, the surgeon can change the threshold for different stages of a single procedure. For instance, the surgeon may input a threshold that is relatively high during a suturing stage of the procedure, but may input a threshold that is relatively low during another stage of the procedure.

If, at 706, the entity determines that the movement is greater than the threshold, then the process 700 proceeds to 708. At 708, the entity outputs a frame with a warning based on the movement. The warning, for instance, includes at least one of a visual alert (e.g., a highlight and/or pop-up), an audio alert, a haptic alert, or the like. On the other hand, if the entity determines that the movement is not greater than the threshold at 706, then the process 700 proceeds to 710. At 710, the entity outputs a frame depicting the instrument in the surgical scene without the indication that the movement is dangerous. In some examples, process 700 can be performed repeatedly, thereby monitoring the surgical environment in real-time.

In some cases, the entity may selectively output the warning if the instrument is of a type that is configured to pierce tissue. For example, the entity may determine that the instrument is a scalpel, scissors, or some other type of instrument configured to pierce tissue before outputting the warning at 708.

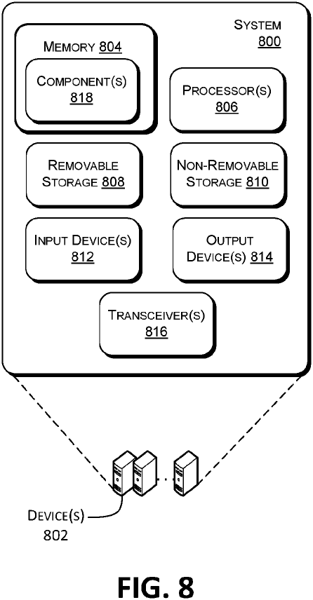

FIG. 8 illustrates an example of a system 800 configured to perform various functions described herein. In various implementations, the system 800 is implemented by one or more computing devices 801, such as servers. The system 800 includes any of memory 804, processor(s) 806, removable storage 808, non-removable storage 810, input device(s) 812, output device(s) 814, and transceiver(s) 816. The system 800 may be configured to perform various methods and functions disclosed herein.

The memory 804 may include component(s) 818. The component(s) 818 may include at least one of instruction(s), program(s), database(s), software, operating system(s), etc. In some implementations, the component(s) 818 include instructions that are executed by processor(s) 806 and/or other components of the device 800. For example, the component(s) 818 include instructions for executing functions of a surgical robot (e.g., the surgical robot 112), a console (e.g., the console 114), a monitor (e.g., the monitor 116), an augmentation system (e.g., the augmentation system 118), or any combination thereof.

In some embodiments, the processor(s) 806 include a central processing unit (CPU), a graphics processing unit (GPU), or both CPU and GPU, or other processing unit or component known in the art.

The device 800 may also include additional data storage devices (removable and/or non-removable) such as, for example, magnetic disks, optical disks, or tape. Such additional storage is illustrated in FIG. 8 by removable storage 808 and non-removable storage 810. Tangible computer-readable media can include volatile and non-volatile, removable and non-removable media implemented in any method or technology for storage of information, such as computer readable instructions, data structures, program modules, or other data. The memory 804, the removable storage 808, and the non-removable storage 810 are all examples of computer-readable storage media. Computer-readable storage media include, but are not limited to, Random Access Memory (RAM), Read-Only Memory (ROM), Electrically Erasable Programmable Read-Only Memory (EEPROM), flash memory, or other memory technology, Compact Disk Read-Only Memory (CD-ROM), Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the system 800. Any such tangible computer-readable media can be part of the system 800.

The system 800 may be configured to communicate over a telecommunications network using any common wireless and/or wired network access technology. Moreover, the device 800 may be configured to run any compatible device Operating System (OS), including but not limited to, Microsoft Windows Mobile, Google Android, Apple iOS, Linux Mobile, as well as any other common mobile device OS.

The system 800 also can include input device(s) 812, such as a keypad, a cursor control, a touch-sensitive display, voice input device, etc., and output device(s) 814 such as a display, speakers, printers, etc. These devices are well known in the art and need not be discussed at length here. In some cases, the input device(s) 812 include at least one of controls (e.g., the controls 124 described above with reference to FIG. 1), a scope (e.g., a scope included in the tools 110 described above with reference to FIG. 1), or sensors (e.g., sensors included in the surgical robot 112 and/or tools 110 of the surgical robot 112). In some examples, the output device(s) 814, include at least one display (e.g., the console display 122 and/or the monitor display 126), a surgical robot (e.g., the surgical robot 112), arms (e.g., arms 120), tools (e.g., the tools 110), or the like.

As illustrated in FIG. 8, the system 800 also includes one or more wired or wireless transceiver(s) 816. For example, the transceiver(s) 816 can include a network interface card (NIC), a network adapter, a Local Area Network (LAN) adapter, or a physical, virtual, or logical address to connect to various network components, for example. To increase throughput when exchanging wireless data, the transceiver(s) 816 can utilize multiple-input/multiple-output (MIMO) technology. The transceiver(s) 816 can comprise any sort of wireless transceivers capable of engaging in wireless (e.g., radio frequency (RF)) communication. The transceiver(s) 816 can also include other wireless modems, such as a modem for engaging in Wi-Fi, WiMAX, Bluetooth, infrared communication, and the like. The transceiver(s) 816 may include transmitter(s), receiver(s), or both.

### Example 1 - Sample Process for Predicting Bleeding in a Surgical Scene

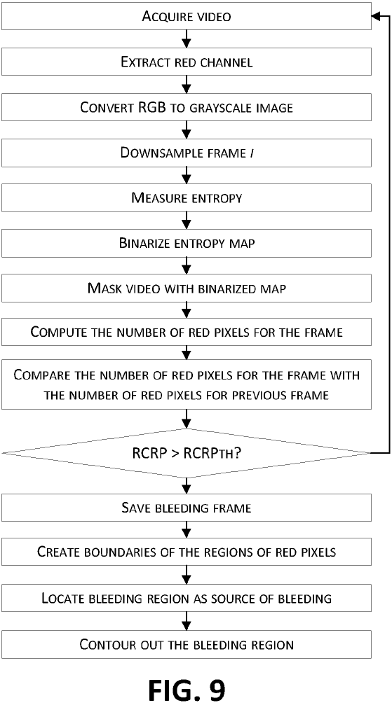

FIG. 9 illustrates a flow chart of a sample process that can be used to predict and/or identify and locate bleeding, such as arterial bleeding. The problem of bleeding detection is modeled as random movement detection. First, the process includes reading the video scene frame by frame. In next step, the local entropy of different blocks of the image was computed and the entropy map of that frame was generated. The entropy map represents the grayscale image with lower intensity for regions that contain more uniform (less information / higher homogeneity) and higher intensity for the regions with more texturized (or more information). In the next step, the entropy map is binarized, and the total number of low-entropy pixels is computed. The low-entropy pixels that are also red pixels correspond to bleeding.

The low-entropy pixels that are also white pixels correspond to tools within the frame. The value of the low-entropy white pixels is used as a quantified indicator for measuring the homogeneity of different regions of the frame. This value can be compared to the value of the number of low-entropy white pixels from the prior frame in order to identify a motion of a tool between the frames. If the motion is greater than a particular threshold, then the frame can be identified as being a frame with abrupt movement of surgical tools. Such abrupt movement can be predictive of future bleeding. The regions of low-entropy white pixels in that frame can be contoured out and overlaid on the original video frame to provide a better visualization to the surgeon of where they need to be more careful.

There are two types of homogeneous regions within the surgery scene: uniform regions that are formed by bleeding and uniform regions that are formed by surgical tools. Various examples described herein identify and track the second category of these homogeneous regions and the rate of changes of their areas and their speed of displacement of their locations.

Various examples can be used to warn surgeons about accidental tool and/or camera movement, which could lead to tissue damage in the patient. The process can be used in the design of predictive and preventive systems for managing hemorrhaging during robotic surgery. It can be crucial to have an artificial vision system that can monitor the movements of surgical tools and warn surgeons about their abrupt movement of surgical instruments. It can also predict the likelihood of sudden bleeding (or other adverse events). The process is capable of this because there is a correlation between the sudden movement of a surgical tool and the occurrence of arterial bleeding. Since the process can distinguish between change in local entropy of the scene introduced by the abrupt movement of surgical instruments and/or the camera, it can be exploited as a warning mechanism to notify surgeons about the way in which they move the surgical tools. Such warnings could prevent the occurrence of bleeding. Furthermore, the process can be utilized to improve the learning curve for new surgeons by informing their movements and increasing their dexterity.

Abrupt movement of surgical tools is one of source of unexpected bleeding during robotic and laparoscopic surgery. The concept of entropy can be utilized to detect the abrupt movement of surgical tools. Abrupt movements of surgical tools resemble a stochastic event, which decreases the quantity of information encoded within the surgical scene. For this work, temporal changes in local entropy can be utilized to detect the stochastic event of sudden movement of surgical tools, which in the context of computer vision can be modeled as an abrupt increase (or change) in the number low-entropy white pixels within the surgery scene after applying the local entropy filter. White pixels are assumed to depict tools due to the typically high luminosity of tooltips of surgical instruments.

First, the process is used to read the video scene frame by frame. In a next step, the local entropy of different blocks of the image is computed and the local entropy map of that frame is generated. The entropy map represents the grayscale image with higher intensity for regions that contain less uniformity (or more information) and lower intensity for the regions with more homogeneity (or less information). In the next step, the entropy map is binarized, and the total number of low-entropy (e.g., white) pixels is computed. The value of the low-entropy pixels is used as a quantified indicator for measuring the entire homogeneity of the frame. This value is compared to the value of the number of low-entropy pixels from the prior frame. This change indicates the velocity of surgical tools. Acceleration and jerk of surgical tools can be computed in the same way. If an occurrence of abrupt change in the jerk exceeds a certain parameterized value within a predefined duration of time, then the movement will be classified as an abrupt movement.

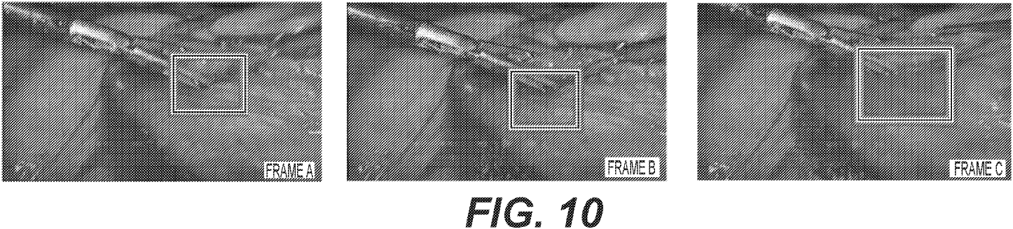

FIG. 10 depicts a change in a surgical scene at a location of arterial bleeding. Frames A through C are consecutive frames in a video of a surgical scene. Unsafe tool movement occurs between Frame A and Frame B. Bleeding begins at Frame C. Therefore, the unsafe tool movement between Frame A and Frame B can be used to predict bleeding before it occurs.

### Example 2: Results for Predicting Bleeding in a Surgical Scene

The process described above in Example 1 was evaluated using 10 sample videos of intraoperative bleeding. The following table describes the duration of each, the frames per second (fps) of each video, the frame at which bleeding actually occurred within each video, the frame at which the bleeding was predicted for each video using this process, and the advance warning (in time) of the bleeding of each video.

TABLE 1

Bleeding Prediction Results

| Video # | Duration (seconds) | fps | Bleeding Time (Frame) | Warning Time (Frame) | Advance Warning (seconds) |
|---|---|---|---|---|---|
| 1 | 20.05 | 60 | 230 | 207 | 0.383333333 |
| 2 | 25.12 | 25 | 237 | 228 | 0.36 |
| 3 | 18.12 | 25 | 335 | 322 | 0.52 |
| 4 | 32.23 | 25 | 227 | 188 | 1.56 |
| 5 | 16.22 | 25 | 94 | 65 | 1.16 |
| 6 | 15.22 | 25 | 164 | 159 | 0.2 |
| 7 | 25.11 | 25 | 244 | 225 | 0.76 |
| 8 | 32.12 | 25 | 479 | 468 | 0.44 |
| 9 | 28.14 | 25 | 372 | 364 | 0.32 |
| 10 | 26.22 | 25 | 294 | 272 | 0.88 |

As shown, this process was successfully utilized to issue a warning before bleeding occurred in all ten of the videos assessed. The sensitivity of this process can be adjusted.

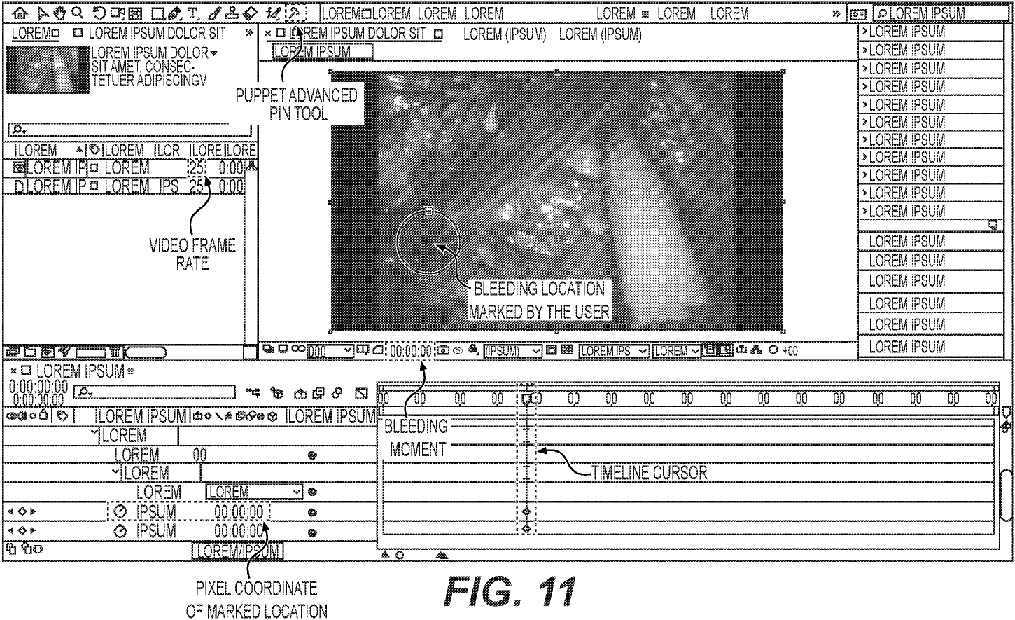

FIG. 11 shows a result of Example 2 following import into Adobe Premier software.

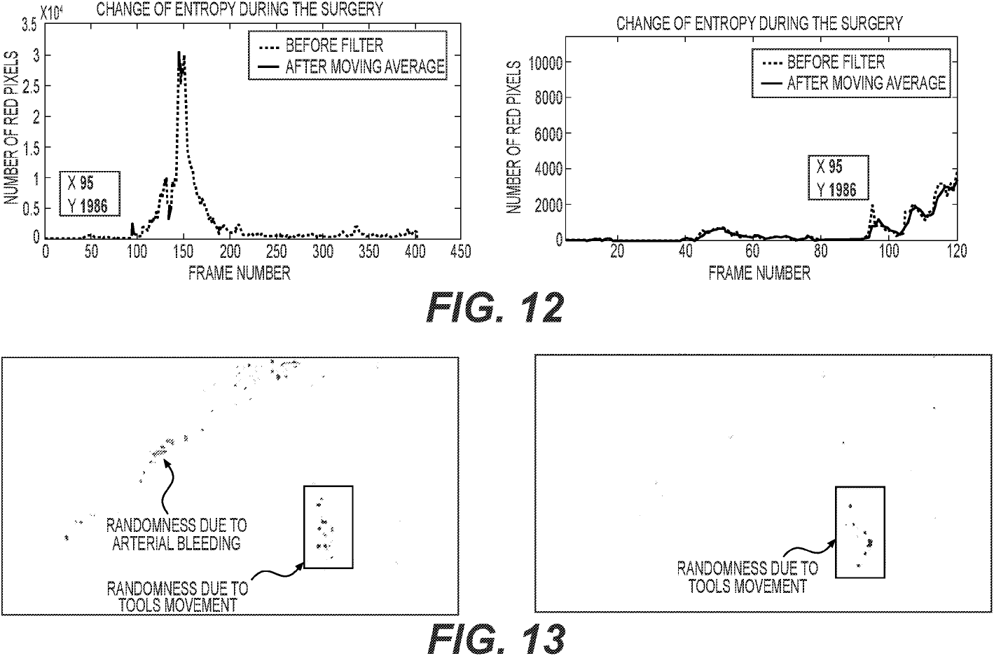

FIG. 12 depicts the temporal entropy within a prerecorded video with arterial bleeding. It can be seen that the first abrupt change in the number of red pixels in the entropy map occurred in frame 95. The right graph is the zoomed version of the left one in the neighborhood of frame number 95. It shows that identifying abrupt changes in the number of red pixels within the local entropy map can be used to detect arterial bleeding within the surgery scene.

FIG. 13 depicts example masked images of two frames in a surgery scene. The masked images include three types of pixels: high-entropy pixels (shown in white), low-entropy red pixels (shown in gray), and low-entropy white pixels (shown in black). The low-entropy white pixels may be tracked in order to identify unsafe tool movement, which can be used to predict bleeding.

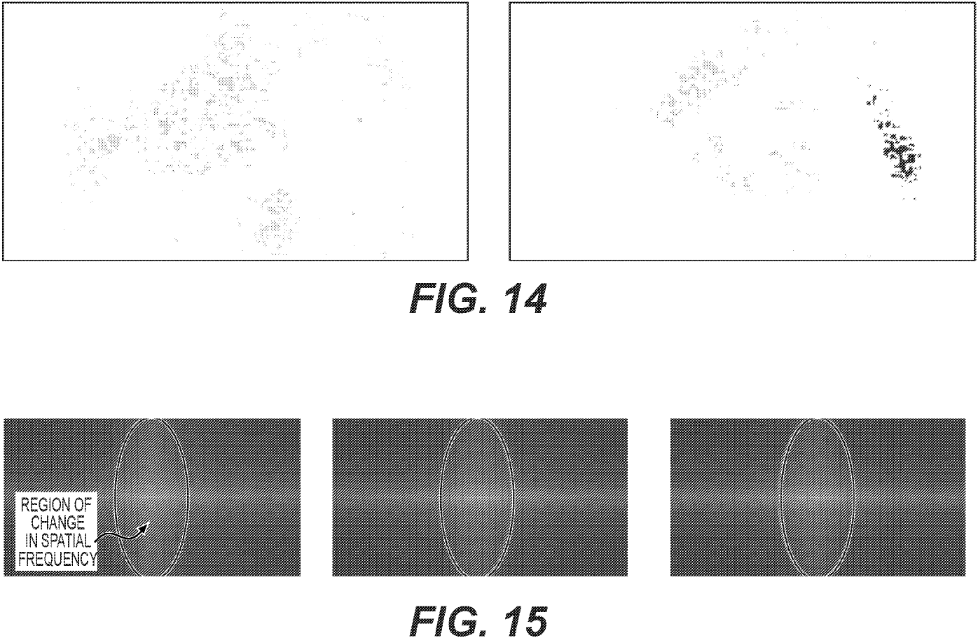

FIG. 14 includes two images demonstrate the effect of arterial bleeding at two time points within a surgery scene. Gray shapes in FIG. 14 represent pixels corresponding to bleeding in the surgical scene. Black shapes in FIG. 14 represent pixels corresponding to tool movement in the surgical scene.

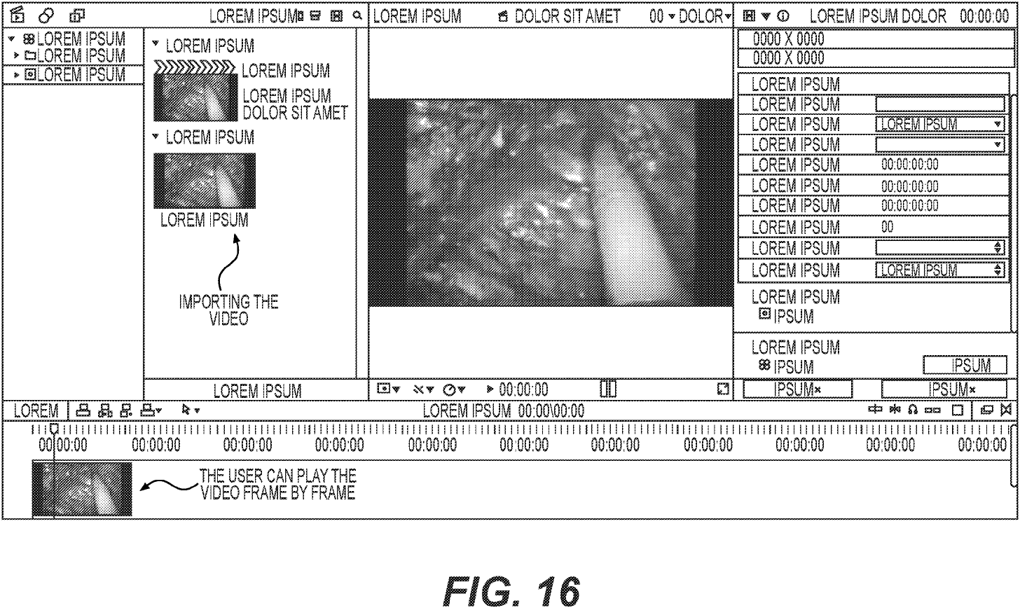

FIG. 15 includes three images comparing the change in the Fourier Transform of the surgery scene.

FIG. 16 illustrates an example of a technique for importing the recorded video into video editing software.

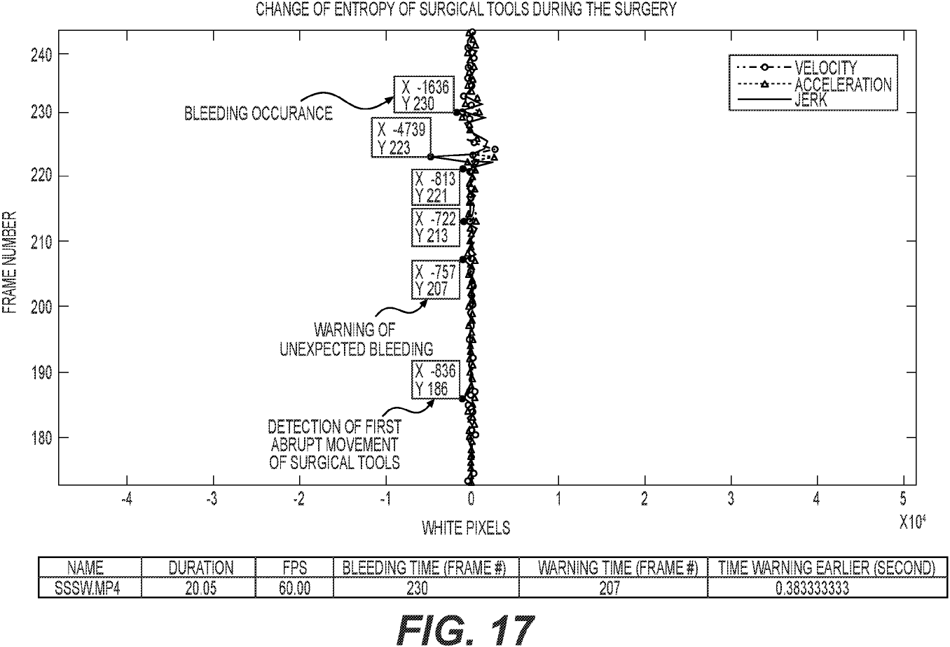

FIG. 17 illustrates a graph showing a change of entropy due to surgical tool movement during a surgery. The graph represents Video #1 in Table 1. As shown, there are distinct spikes in higher order spatio-temporal entropy within frames of the video prior to bleeding being depicted in the video.

### EXAMPLE CLAUSES

1. A method, including: identifying a movement of an instrument; determining that the movement of the instrument exceeds a threshold; and dampening the movement of the instrument based on determining that the movement exceeds the threshold.

2. The method of clause 1, further including: identifying a type of the instrument; and determining that the instrument is configured to cut into tissue based on the type.

3. The method of clause 2, wherein the instrument includes a scalpel or scissors.

4. The method of any one of clauses 1 to 3, wherein identifying the movement of the instrument includes analyzing kinematic data of a surgical robot directing the movement of the instrument.

5. The method of any one of clauses 1 to 4, wherein the movement includes at least one of a velocity of the instrument, an acceleration of the instrument, or a jerk of the instrument.

6. The method of any one of clauses 1 to 5, wherein identifying the movement includes analyzing multiple frames depicting the instrument.

7. The method of clause 6, wherein analyzing the multiple frames includes determining the movement based on a change in entropy of the multiple frames.

8. The method of any one of clauses 1 to 7, wherein identifying the movement of the instrument includes: identifying a first frame depicting the instrument at a first time; identifying a second frame depicting the instrument at a second time, the second time being after the first time; generating a first masked image indicating first entropies of first pixels in the first frame; generating a second masked image indicating second entropies of second pixels in the second frame; determining a change between the first masked image and the second masked image; and identifying the movement based on the change.

9. The method of clause 8, wherein the change corresponds to a velocity of the instrument.

10. The method of clause 8 or 9, wherein the movement includes a velocity of the instrument.

11. The method of any one of clauses 8 to 10, the change being a first change, wherein identifying the movement of the instrument further includes: identifying a third frame depicting the instrument at a third time, the third time being before the second time; generating a third masked image indicating third entropies of third pixels in the third frame; determining a second change between the third masked image and the first masked image; determining a third change between the second change and the first change; and identifying the movement based on the third change.

12. The method of clause 11, wherein the third change corresponds to an acceleration of the instrument.

13. The method of clause 11 or 12, wherein the movement includes an acceleration of the instrument.

14. The method of any one of clauses 11 to 13, wherein identifying the movement of the instrument further includes: identifying a fourth frame depicting the instrument at a fourth time, the fourth time being before the third time; generating a fourth masked image indicating fourth entropies of fourth pixels in the fourth frame; determining a fourth change between the fourth masked image and the third masked image; determining a fifth change between the fourth change and the second change; determining a sixth change between the fifth change and the third change; and identifying the movement based on the sixth change.

15. The method of clause 14, wherein the sixth change corresponds to a jerk of the instrument.

16. The method of clause 14 or 15, wherein the movement includes a jerk of the instrument.

17. The method of any one of clauses 8 to 16, wherein generating the first masked image includes: generating the first entropies by convolving an entropy kernel with a detection window, the first frame including the detection window; generating an entropy map by comparing the first entropies to a threshold; and generating the first masked image by performing pixel-by-pixel multiplication of the entropy map and at least one color channel of the first frame.

18. The method of clause 17, wherein the at least one color channel includes a red color channel.

19. The method of clause 17 or 18, wherein determining the change between the first masked image and the second masked image includes: determining a first number of pixels in the first masked image with values that are under a threshold; determining a second number of pixels in the second masked image with values that are under the threshold; and determining the change by subtracting the second number from the first number.

20. The method of any one of clauses 8 to 19, wherein determining the change between the first masked image and the second masked image includes: determining a first ratio of pixels in the first masked image with values that are greater than a threshold; determining a second ratio of pixels in the second masked image with values that are greater than the threshold; and determining the change by subtracting the second number from the first number.

21. The method of any one of clauses 1 to 20, further including: setting the threshold based on a user input.

22. The method of any one of clauses 1 to 21, wherein dampening the instrument includes at least one of slowing a velocity of the instrument, decelerating the instrument, or reducing a jerk of the instrument.

23. The method of any one of clauses 1 to 22, wherein the instrument includes metal.

24. The method of any one of clauses 1 to 23, further including: identifying a user input corresponding to a directed movement of the instrument, wherein dampening the instrument includes causing an actual movement of the tool at the third time based on the user input, the actual movement being dampened with respect to the directed movement.

25. The method of any one of clauses 1 to 24, further including: outputting a warning based on the movement of the instrument.

26. The method of clause 25, wherein the warning indicates that the movement is dangerous and/or that the movement is predicted to cause bleeding.

27. The method of clause 26, wherein outputting the warning includes outputting at least one of a visual alert, an audio alert, or a haptic alert.

28. The method of clause 26 or 27, wherein outputting the warning includes outputting a visual alert with the second frame, the visual alert overlaying the instrument and/or sensitive tissue within a threshold distance of the instrument.

29. The method of clause 28, further including: identifying the sensitive tissue based on a SLAM analysis of a surgical field that includes the instrument and the sensitive tissue.

30. The method of any one of clauses 1 to 29, further including: training, based on multiple videos depicting tools that cause bleeding, a machine learning model to identify tool movements associated with bleeding, wherein determining that the movement exceeds the threshold includes inputting a video of the movement of the instrument into the machine learning model.

31. The method of any one of clauses 1 to 30, further including: identifying a position of a tissue structure; identifying a position of the instrument; and determining that the position of the tissue structure is within a threshold distance of the position of the instrument.

32. The method of clause 31, wherein identifying the position of the tissue structure includes: generating data indicative of the tissue structure; and determining the position of the tissue structure by performing a SLAM analysis on the data.

33. The method of clause 32, wherein generating the data includes: generating, by a camera, one or more images depicting a surgical scene that includes the tissue structure.

34. The method of clause 32 or 33, wherein generating the data includes: generating, by a 3D scanner, a volumetric scan of a surgical scene that includes the tissue structure.

35. A system, including: at least one processor; and memory storing instructions that, when executed by the at least one processor, cause the system to perform operations including: the method of any one of clauses 1 to 34.

36. The system of clause 35, further including: a scope configured to obtain multiple frames depicting the instrument.

37. The system of clause 36, further including: a display configured to output the multiple frames.

38. The system of any one of clauses 35 to 37, wherein the system is a robotic surgical system.

39. The system of clause 38, further including: a console configured to receive a user input directing the movement of the instrument.

40. A non-transitory computer-readable storage medium encoding instructions to perform the method of any one of clauses 1 to 34.

41. A robotic surgical system, comprising: a camera configured to capture a video of a surgical scene; an instrument in the surgical scene; a console configured to receive a user input directing the movement of the instrument at least one processor; and memory storing instructions that, when executed by the at least one processor, cause the system to perform operations comprising: identifying, in the video, a first frame depicting the instrument at a first time; identifying, in the video, a second frame depicting the instrument at a second time, the second time being after the first time; generating a first masked image indicating first entropies of first pixels in the first frame; generating a second masked image indicating second entropies of second pixels in the second frame; determining a change between the first masked image and the second masked image; and identifying a movement of the instrument based on the change; determining that the movement of the instrument exceeds a threshold; and based on determining that the movement of the instrument exceeds the threshold: outputting a warning indicating that the movement is dangerous; and dampening the movement.

42. The robotic surgical system of clause 41, wherein the first pixels comprise white pixels in the first frame, and wherein the second pixels comprise white pixels in the second frame.

43. The robotic surgical system of clause 41 or 42, the threshold being a first threshold, wherein generating the first masked image comprises: generating the first entropies by convolving an entropy kernel with a detection window in the first frame; generating a first entropy mask by comparing the first entropies to a second threshold; and generating the first masked image by performing pixel-by-pixel multiplication of the first entropy mask and at least one color channel of the first frame, and wherein generating the second masked image comprises: generating the second entropies by convolving the entropy kernel with a detection window in the second frame; generating a second entropy mask by comparing the second entropies to the second threshold; and generating the second masked image by performing pixel-by-pixel multiplication of the second entropy mask and at least one color channel of the second frame.

### CONCLUSION

As will be understood by one of ordinary skill in the art, each embodiment disclosed herein can comprise, consist essentially of or consist of its particular stated element, step, or component. Thus, the terms “include” or “including” should be interpreted to recite: “comprise, consist of, or consist essentially of.” As used herein, the transition term “comprise” or “comprises” means has, but is not limited to, and allows for the inclusion of unspecified elements, steps, or components, even in major amounts. The transitional phrase “consisting of” excludes any element, step, or component not specified. The transition phrase “consisting essentially of” limits the scope of the embodiment to the specified elements, steps, or components and to those that do not materially affect the embodiment. The term “based on” should be interpreted as “based at least partly on,” unless otherwise specified.

Unless otherwise indicated, all numbers expressing quantities of properties used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing this disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the present disclosure otherwise claimed. No language in the specification should be construed as indicating any non-claimed element essential to the practice of the techniques described herein.

Groupings of alternative elements or implementations disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors. Of course, variations on these described embodiments will become apparent to those of ordinary skill in the art upon reading the foregoing description. The inventor expects skilled artisans to employ such variations as appropriate, and the inventors intend for the techniques disclosed herein to be practiced otherwise than specifically described herein. Accordingly, the scope of the claims of this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

In closing, it is to be understood that the embodiments of the disclosure are illustrative of the principles of the present invention. Other modifications that can be employed are within the scope of the implementations described herein. Thus, by way of example, but not of limitation, alternative configurations of the present disclosure can be utilized in accordance with the teachings herein. Accordingly, the present disclosure is not limited to that precisely as shown and described.

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings and/or examples making apparent to those skilled in the art how the several forms of the disclosure can be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster’s Dictionary, 3rd Edition.

What is claimed is:

1. A robotic surgical system, comprising:
a camera configured to capture a video of a surgical scene;
an instrument in the surgical scene;
a console configured to receive a user input directing the movement of the instrument
at least one processor; and
memory storing instructions that, when executed by the at least one processor, cause the system to perform operations comprising:
identifying, in the video, a first frame depicting the instrument at a first time;
identifying, in the video, a second frame depicting the instrument at a second time, the second time being after the first time;
generating a first masked image indicating first entropies of first pixels in the first frame;
generating a second masked image indicating second entropies of second pixels in the second frame;
determining a change between the first masked image and the second masked image; and
identifying a movement of the instrument based on the change;
determining that the movement of the instrument exceeds a threshold; and
based on determining that the movement of the instrument exceeds the threshold:
outputting a warning indicating that the movement is dangerous; and
dampening the movement.

2. The robotic surgical system of claim 1, wherein the first pixels comprise white pixels in the first frame, and
wherein the second pixels comprise white pixels in the second frame.

3. The robotic surgical system of claim 1, the threshold being a first threshold, wherein generating the first masked image comprises:
generating the first entropies by convolving an entropy kernel with a detection window in the first frame;
generating a first entropy mask by comparing the first entropies to a second threshold; and
generating the first masked image by performing pixel-by-pixel multiplication of the first entropy mask and at least one color channel of the first frame, and
wherein generating the second masked image comprises:
generating the second entropies by convolving the entropy kernel with a detection window in the second frame;
generating a second entropy mask by comparing the second entropies to the second threshold; and
generating the second masked image by performing pixel-by-pixel multiplication of the second entropy mask and at least one color channel of the second frame.

4. A method, comprising:
identifying a movement of an instrument;
determining that the movement of the instrument exceeds a threshold; and
dampening the movement of the instrument based on determining that the movement exceeds the threshold.

5. The method of claim 4, further comprising:
identifying a type of the instrument; and
determining that the instrument is configured to cut into tissue based on the type.

6. The method of claim 5, wherein the instrument comprises a scalpel or scissors.

7. The method of claim 4, wherein identifying the movement of the instrument comprises analyzing kinematic data of a surgical robot directing the movement of the instrument.

8. The method of claim 4, wherein the movement comprises at least one of a velocity of the instrument, an acceleration of the instrument, or a jerk of the instrument.

9. The method of claim 4, wherein identifying the movement comprises analyzing multiple frames depicting the instrument.

10. The method of claim 9, wherein analyzing the multiple frames comprises determining the movement based on a change in entropy of the multiple frames.

11. The method of claim 4, wherein identifying the movement of the instrument comprises:
identifying a first frame depicting the instrument at a first time;
identifying a second frame depicting the instrument at a second time, the second time being after the first time;
generating a first masked image indicating first entropies of first pixels in the first frame;
generating a second masked image indicating second entropies of second pixels in the second frame;
determining a change between the first masked image and the second masked image; and
identifying the movement based on the change.

12. The method of claim 11, wherein the change corresponds to a velocity of the instrument.

13. The method of claim 11, wherein the movement comprises a velocity of the instrument.

14. The method of claim 11, the change being a first change, wherein identifying the movement of the instrument further comprises:
identifying a third frame depicting the instrument at a third time, the third time being before the second time;
generating a third masked image indicating third entropies of third pixels in the third frame;
determining a second change between the third masked image and the first masked image;
determining a third change between the second change and the first change; and
identifying the movement based on the third change.

15. The method of claim 14, wherein the third change corresponds to an acceleration of the instrument.

16. The method of claim 14, wherein the movement comprises an acceleration of the instrument.

17. The method of claim 14, wherein identifying the movement of the instrument further comprises:
identifying a fourth frame depicting the instrument at a fourth time, the fourth time being before the third time;
generating a fourth masked image indicating fourth entropies of fourth pixels in the fourth frame;
determining a fourth change between the fourth masked image and the third masked image;
determining a fifth change between the fourth change and the second change;
determining a sixth change between the fifth change and the third change; and
identifying the movement based on the sixth change.

18. The method of claim 17, wherein the sixth change corresponds to a jerk of the instrument.

19. The method of claim 17, wherein the movement comprises a jerk of the instrument.

20. The method of claim 11, wherein generating the first masked image comprises:
generating the first entropies by convolving an entropy kernel with a detection window, the first frame comprising the detection window;
generating an entropy mask by comparing the first entropies to a threshold; and
generating the first masked image by performing pixel-by-pixel multiplication of the entropy mask and at least one color channel of the first frame.

21. The method of claim 20, wherein the at least one color channel is a red color channel.

22. The method of claim 20, wherein determining the change between the first masked image and the second masked image comprises:
determining a first number of pixels in the first masked image with values that are under a threshold;
determining a second number of pixels in the second masked image with values that are under the threshold; and
determining the change by subtracting the second number from the first number.

23. The method of claim 11, wherein determining the change between the first masked image and the second masked image comprises:
determining a first ratio of pixels in the first masked image with values that are greater than a threshold;
determining a second ratio of pixels in the second masked image with values that are greater than the threshold; and
determining the change by subtracting the second number from the first number.

24. The method of claim 4, further comprising:
setting the threshold based on a user input.

25. The method of claim 4, wherein dampening the instrument comprises at least one of slowing a velocity of the instrument, decelerating the instrument, or reducing a jerk of the instrument.

26. The method of claim 4, wherein the instrument comprises metal.

27. The method of claim 4, further comprising:
identifying a user input corresponding to a directed movement of the instrument,
wherein dampening the instrument comprises causing an actual movement of the tool at the third time based on the user input, the actual movement being dampened with respect to the directed movement.

28. The method of claim 4, further comprising:
outputting a warning based on the movement of the instrument.

29. The method of claim 28, wherein the warning indicates that the movement is dangerous and/or that the movement is predicted to cause bleeding.

30. The method of claim 29, wherein outputting the warning comprises outputting at least one of a visual alert, an audio alert, or a haptic alert.

31. The method of claim 29, wherein outputting the warning comprises outputting a visual alert with the second frame, the visual alert overlaying the instrument and/or sensitive tissue within a threshold distance of the instrument.

32. The method of claim 31, further comprising:
identifying the sensitive tissue based on a SLAM analysis of a surgical field that comprises the instrument and the sensitive tissue.

33. The method of claim 4, further comprising:
training, based on multiple videos depicting tools that cause bleeding, a machine learning model to identify tool movements associated with bleeding,
wherein determining that the movement exceeds the threshold comprises inputting a video of the movement of the instrument into the machine learning model.

34. The method of claim 4, further comprising:
identifying a position of a tissue structure;
identifying a position of the instrument; and
determining that the position of the tissue structure is within a threshold distance of the position of the instrument.

35. The method of claim 34, wherein identifying the position of the tissue structure comprises:
generating data indicative of the tissue structure; and
determining the position of the tissue structure by performing a SLAM analysis on the data.

36. The method of claim 35, wherein generating the data comprises:
generating, by a camera, one or more images depicting a surgical scene that comprises the tissue structure.

37. The method of claim 35, wherein generating the data comprises:
generating, by a 3D scanner, a volumetric scan of a surgical scene that comprises the tissue structure.

38. A system, comprising:
at least one processor; and
memory storing instructions that, when executed by the at least one processor, cause the system to perform operations comprising:
identifying a movement of an instrument;
determining that the movement of the instrument exceeds a threshold; and
dampening the movement of the instrument based on determining that the movement exceeds the threshold.

39. The system of claim 38, further comprising:
a scope configured to obtain multiple frames depicting the instrument.

40. The system of claim 39, further comprising:
a display configured to output the multiple frames.

41. The system of claim 38, wherein the system is a robotic surgical system.

42. The system of claim 41, further comprising:
a console configured to receive a user input directing the movement of the instrument.
